(19) United States
(12) Patent Application Publication
(10) Pub. No.: US 2023/0035080 A1
(43) Pub. Date: Feb. 2, 2023

(54) TWO-DIMENSIONAL (2D) NANOCOMPOSITE, PREPARATION METHOD, AND USE THEREOF

(71) Applicants: NINGBO INSTITUTE OF MATERIALS TECHNOLOGY & ENGINEERING, CHINESE ACADEMY OF SCIENCES, Ningbo (CN); Ningbo Cixi Institute of BioMedical Engineering, Ningbo (CN)

(72) Inventors: Aiguo WU, Ningbo (CN); Tianxiang CHEN, Ningbo (CN); Qianlan FANG, Ningbo (CN)

(73) Assignees: NINGBO INSTITUTE OF MATERIALS TECHNOLOGY & ENGINEERING, CHINESE ACADEMY OF SCIENCES, Ningbo (CN); Ningbo Cixi Institute of BioMedical Engineering, Ningbo (CN)

(21) Appl. No.: 17/790,145

(22) PCT Filed: Dec. 29, 2020
(86) PCT No.: PCT/CN2020/140788

§ 371 Date: Jun. 30, 2022

(30) Foreign Application Priority Data
Dec. 31, 2019 (CN) 201911411094.0

(51) Int. Cl. A61K 41/00

(52) U.S. Cl. CPC A61K 41/0057; B82Y 5/00

(57) ABSTRACT

A nanocomposite includes an oxygen vacancy-containing BiOX particle and a coating, where the coating is a biocompatible material. Under near-infrared (NIR) irradiation, the nanocomposite has a photothermal conversion efficiency of greater than or equal to 10%. Under NIR irradiation, the nanocomposite degrades 1,3-diphenylisobenzofuran (DPBF) at a rate of higher than or equal to 0.1 mmol/h. BiOX may be BiOF, BiOCl, BiOBr, BiOI, or BiOAt. A preparation method and a use of the nanocomposite are further provided. The nanocomposite is a bismuth oxyhalide nanomaterial with different numbers of oxygen vacancies and can be used for the photothermal therapy (PTT) of a tumor and for the integrated tumor diagnosis and treatment. The nanocomposite leads to an excellent therapeutic effect under the guidance of multi-modality imaging, and has excellent computed tomography (CT) imaging and photoacoustic imaging (PAI) performance.

### CROSS REFERENCE TO THE RELATED APPLICATIONS

This application is the national phase entry of International Application No. PCT/CN2020/140788, filed on Dec. 29, 2020, which is based upon and claims priority to Chinese Patent Application No. 201911411094.0, filed on Dec. 31, 2019, the entire contents of which are incorporated herein by reference.

### TECHNICAL FIELD

The present disclosure relates to the field of materials, and in particular to two-dimensional (2D) bismuth oxyhalide nanomaterials with different numbers of oxygen vacancies, a preparation method, and a use thereof.

### BACKGROUND

Malignant tumors have been known as the leading cause of death. According to incomplete statistics, 9.6 million people die of cancer, and 18.1 million new confirmed cases of cancer are reported worldwide every year. In view of the high incidence rate and high mortality rate of cancer, researchers around the world have always been committed to developing accurate and rapid diagnosis and treatment methods for cancer. Traditional cancer treatment methods include chemotherapy, radiotherapy, and surgery, but these traditional treatment methods may make patients suffer from serious side effects. Radiotherapy and chemotherapy are less targeted and will cause serious damage to normal tissues and organs. Surgery is not effective for widely-metastatic malignancies. The failure of these treatments drives researchers to constantly study the development of accurate and effective treatment strategies for cancer. Emerging cancer treatment methods include, but are not limited to, immunotherapy, gene therapy, photodynamic therapy (PDT), photothermal therapy (PTT), and targeted therapy, which have improved the treatment outcomes of many patients to varying degrees. Phototherapy is a novel non-invasive treatment method with high accuracy and efficiency and has attracted extensive attention of researchers.

Phototherapy is currently the least invasive therapy and mainly includes PDT and PTT. PDT is a clinically approved minimally invasive treatment that can selectively produce cytotoxic activity in tumor cells. In PDT, a photosensitizing agent is delivered to a tumor area and then irradiated with light at a wavelength corresponding to an absorbance band of the photosensitizing agent. In the presence of oxygen, singlet oxygen, superoxide radicals, or hydroxyl radicals are generated to cause the direct death of tumor cells, cause damage to the microvasculature, and induce local inflammatory responses. PTT refers to the interaction of incident light with free electrons of nanoparticles. When a wavelength of the incident light is resonantly coupled with a vibration frequency of the free electrons, a thermal effect occurs due to the generation of surface plasmon resonance (SPR). The thermal effect can directly change the permeability of a biological membrane, result in a malformed and disorganized microvascular network within a tumor, directly cause a tumor area to undergo slow heat dissipation and become a natural heat reservoir, and hinder the growth of tumor vasculature by inhibiting the expression of a tumor-derived vascular endothelial growth factor (VEGF) and its receptor, thereby inhibiting the growth and metastasis of a tumor.

However, using only PTT is not effective against a tumor because the generated heat is unevenly distributed and the heat generated around great vessels is easily dissipated quickly, making it difficult to completely remove a tumor and causing tumor recurrence in some areas. PDT as a treatment by itself also has limited treatment effect. A toxicity mechanism of PDT is mainly to convert oxygen in a tissue into reactive oxygen species (ROS), however, a tumor tissue usually presents severe hypoxia. The generation of ROS can stimulate the activation of an anti-oxidative stress (AOS) system of tumor cells, resulting in PDT resistance. In order to make up for the shortcomings of using a single phototherapy treatment, researchers are constantly exploring a new nanomaterial for enhancing the synergistic interaction of an anti-cancer treatment using a combination of PDT and PTT. It is desirable that this material can serve as both a photosensitizing agent carrier and a heat source, so that a treatment plan combining PDT and PTT is realized. However, the current research on the combined therapy remains unable to provide a solution to the following shortcomings:

1. Although PTT excited by near-infrared (NIR) light has been reported in many literature sources, the research on PDT excited by NIR light remains stagnant. Two kinds of laser are used for irradiation in many studies, but they result in unnecessary side effects.

2. In order to achieve NIR-triggered synchronous photothermal and photodynamic effects, many researchers use upconversion materials (such as lanthanides) to convert NIR light into ultraviolet (UV) or visible light with a short wavelength, thereby exciting photosensitive molecules to generate ROS. However, the high biological toxicity of such materials limits their further application.

3. Most of the nanomaterials for the combined therapy are composed of two or more materials and have disadvantages, such as complex synthetic route, possible additional biological toxicity, and the need for multi-irradiation. Therefore, there is an urgent need for a single-component nanomaterial that possesses both PDT and PTT functions under NIR laser irradiation, which can work synergistically with an immune checkpoint inhibitor and advances the progress of the research on the combined therapy.

In recent years, inorganic bismuth-based materials have been widely studied as a novel anti-tumor photosensitizing agent in the diagnosis and treatment of cancer due to their unique physical and chemical properties, strong NIR absorption capacity, and excellent photothermal conversion performance. Due to the high X-ray absorption coefficient of bismuth, bismuth-based nanomaterials can also be used for computed tomography (CT) imaging and photoacoustic imaging (PAI). In addition, bismuth-based materials have the advantages of simple synthetic method, low cost, long in vivo circulation time, and excellent dispersibility. Although bismuth-based nanomaterials have been extensively studied in the catalysis field and it has been discovered that nanomaterials, such as Bi₂Se₃ and Bi₂S₃, can achieve a prominent photothermal effect in mouse tumor models, the biomedical research on bismuth-based nanomaterials is still in its infancy stage. The cytotoxicity and instability of bismuth-based nanoparticles have always been the main problems limiting their application in the biomedical field.

In recent years, the use of BiOX (X=F, Cl, Br, I, or At) nanomaterials in the diagnosis and treatment of tumors has gradually attracted the attention of researchers. With special electronic structures, BiOX nanomaterials have been reported to have a strong laser-induced ROS production ability, and Bi 6s and O 2p can form a prominent hybrid valence band. The hybridization of Bi 6s and O 2p makes the valence band dispersed to a large extent, which is conducive to the migration of light-induced holes in the valence band and the progress of an oxidation reaction. Therefore, ultrathin nanosheets of such nanomaterials have received more attention in energy conversion and storage. An ultrathin nanosheet with a 2D structure makes photoexcited electron-hole pairs (EHPs) reach a surface more easily than EHPs generated in vivo, which reduces the recombination chance. The atomic thickness and surface distortion and defect of an ultrathin 2D crystal play an important role in the electronic structural modification and performance improvement of the crystal. However, most of the BiOX nanomaterials have a wide band gap, can only be excited by high-energy UV light or X-rays, and are only used in the radiotherapy of tumors, which inevitably causes damage to healthy tissues. Moreover, due to the wide band gap, BiOX nanomaterials do not possess photothermal properties. Inspired by the above research, the present disclosure proposes an ultrathin BiOX nanosheet with a large number of surface/subsurface defects, which introduces the photothermal properties while retaining the photoexcited ROS production ability to enable the efficient diagnosis and treatment of tumors.

X-ray CT is a main clinical diagnosis method with high resolution and no depth limitation. However, diagnosing tumors using only the single method of CT imaging is less desirable and has various inherent limitations, such as poor contrast in soft tissues, low flux, and high ionizing radiation. Therefore, the use of a combination of CT and PAI techniques for diagnosis may combine the advantages of the two techniques while avoiding their disadvantages. The ultrathin BiOX nanosheet has ideal photothermal and photodynamic effects in PAI/CT dual-modality imaging. These features allow BiOX nanomaterials to be “accurately targeting weapons”, which provides a new platform for destroying in vivo solid tumors and preventing tumor metastasis.

### SUMMARY

According to an aspect of the present disclosure, a series of nanocomposites are provided. The nanocomposites can be used for the integrated diagnosis and treatment of a tumor and have excellent dispersibility in water, low biological toxicity, and excellent crystallinity. Therefore, the nanocomposites work well in the diagnosis and treatment of cancer and can reduce the toxic and side effects.

The nanocomposite includes an oxygen vacancy-containing BiOX particle and a coating, where the coating is a biocompatible material.

Under NIR irradiation, the nanocomposite has a photothermal conversion efficiency of greater than or equal to 10%. Under NIR irradiation, the nanocomposite degrades 1,3-diphenylisobenzofuran (DPBF) at a rate of higher than or equal to 0.1 mmol/h.

BiOX is at least one compound selected from the group consisting of BiOF, BiOCl, BiOBr, BiOI, and BiOAt.

Optionally, a content of BiOCl in the BiOX particle may be greater than or equal to 50 wt %, preferably greater than or equal to 60 wt %, more preferably greater than or equal to 70 wt %, more preferably greater than or equal to 80 wt %, more preferably greater than or equal to 90 wt %, and most preferably greater than or equal to 95 wt % (such as 99 wt %).

Optionally, a proportion of the oxygen vacancy in the oxygen vacancy-containing BiOX particle may be 20% or higher.

Optionally, a proportion of the oxygen vacancy in the oxygen vacancy-containing BiOX particle may be 20% to 30%. The BiOX particle with an oxygen vacancy proportion of 20% to 30% is grey.

Optionally, a proportion of the oxygen vacancy in the oxygen vacancy-containing BiOX particle may be 40% or higher. The BiOX particle with an oxygen vacancy proportion of 40% or higher is black.

Optionally, under NIR irradiation, the nanocomposite may have a photothermal conversion efficiency of greater than or equal to 10%.

Optionally, under NIR irradiation, the nanocomposite may have a photothermal conversion efficiency of greater than or equal to 40%.

Optionally, under NIR-II irradiation, the nanocomposite may degrade DPBF at a rate of higher than or equal to 1 mmol/h.

Optionally, the nanocomposite may have a CT signal grey value of greater than or equal to 100, preferably greater than or equal to 800, and more preferably greater than or equal to 1,400.

Optionally, the nanocomposite may have a PAI signal grey value of greater than or equal to 100, preferably greater than or equal to 400, and more preferably greater than or equal to 800.

Optionally, the BiOX particle may have a particle size of greater than or equal to 0.1 nm.

Optionally, the BiOX particle may have a particle size of 0.1 nm to 500 nm, preferably 20 nm to 250 nm, more preferably 30 nm to 200 nm, more preferably 70 nm to 130 nm, more preferably 100 nm to 125 nm, and most preferably 110 nm to 125 nm.

Optionally, the BiOX particles with oxygen vacancies may include oxygen vacancies.

Optionally, the BiOX particles with oxygen vacancies may be a 2D layered crystal.

Optionally, the nanocomposite may be a 2D nanocomposite in which an oxygen vacancy-containing BiOCl particle serves as a core and is coated with a biocompatible material.

Optionally, the 2D nanocomposite can be stably dispersed in an aqueous solution.

Optionally, after the 2D nanocomposite is dispersed in water or 0.9% normal saline (NS) for 60 d, a change in average particle size of the 2D nanocomposite may be smaller than or equal to 20%.

Optionally, after the 2D nanocomposite is dispersed in water or 0.9% NS for 30 d to 40 d, preferably 40 d to 50 d, and more preferably 50 d to 60 d, a change in the average particle size of the 2D nanocomposite may be smaller than or equal to 15%, more preferably smaller than or equal to 10%, more preferably smaller than or equal to 5%, and most preferably smaller than or equal to 3%.

Optionally, the 2D nanocomposite may have a D50 of 120 nm, preferably 100 nm, and more preferably 80 nm.

Optionally, based on a total number of particles of the 2D nanocomposite, particle sizes of 70% of the particles may be within a range of D50 of the 2D nanocomposite ±20%.

Optionally, based on a total number of particles of the 2D nanocomposite, particle sizes of 80% (preferably 85%, more preferably 90%, and most preferably 93%) of the particles may be within a range of D50 of the 2D nanocomposite ±15% (preferably ±10% and more preferably ±8%).

Optionally, the coating may be at least one selected from the group consisting of a polysaccharide and a derivative thereof, an amino acid and a derivative thereof, a polyol and a derivative thereof, a polymer polyol, and polyacrylic acid (PAA) and a derivative thereof.

Optionally, the coating may be at least one selected from the group consisting of polyethylene glycol (PEG) and a derivative thereof, mannitol, modified chitosan, dextran, carboxyl dextran, liposome, albumin, tetraethylorthosilicate (TEOS), PAA, KH560, KH550, F127, CO-520, diethyl enetriaminepentaacetic acid (DTPA), meglumine, arginine, polyglutamic acid (PGA), and polypeptide.

Optionally, a mass ratio of the BiOX particle to the coating may be 100:1 to 1:1.

Optionally, a mass ratio of the BiOX particle to the coating may be 100:1 to 10:1.

The present disclosure also provides a preparation method of the nanocomposite with excellent performance, which is environmentally friendly, safe, reliable, simple in process, low in cost, and high in yield.

The preparation method of the nanocomposite includes the following steps:

a) acquiring the oxygen vacancy-containing BiOX particle; and

b) coating the oxygen vacancy-containing BiOX particle to obtain the nanocomposite.

Optionally, the preparation method of the nanocomposite may include the following steps:

a1) thoroughly mixing a Bi-containing oxycompound, a Bi-containing halide, and a solvent, and allowing a solvothermal reaction to obtain an oxygen vacancy-containing BiOX particle I, where a proportion of the oxygen vacancy in the oxygen vacancy-containing BiOX particle I is 20% to 30%; and

b) mixing a dispersion I of the oxygen vacancy-containing BiOX particle I with a coating or coating precursor-containing solution, and allowing a reaction to obtain the nanocomposite.

Optionally, the preparation method of the nanocomposite may include the following steps:

a1) thoroughly mixing a Bi-containing oxycompound, a Bi-containing halide, and a solvent, and allowing a solvothermal reaction to obtain an oxygen vacancy-containing BiOX particle I, where a proportion of the oxygen vacancy in the oxygen vacancy-containing BiOX particle I is 20% to 30%;

a2) subjecting a dispersion I of the oxygen vacancy-containing BiOX particle I to a reduction treatment to obtain a dispersion II of an oxygen vacancy-containing BiOX particle II, where a proportion of the oxygen vacancy in the oxygen vacancy-containing BiOX particle II is 40% or higher; and

b) mixing the dispersion II with a coating or coating precursor-containing solution, and allowing a reaction to obtain the nanocomposite.

Optionally, in step a1), a mass ratio of the Bi-containing oxycompound to the Bi-containing halide may be (10-1):(0.1-1).

Optionally, in step a1), the Bi-containing oxycompound may be at least one selected from the group consisting of Bi₂O₃, Bi₂(SO₄)₃, Bi(NO₃)₃.5H₂O, BiPO₄, BiH(PO₃)₂, BiH₂PO₃, Bi₂(CO₃)₃, Bi₂(SO₄)₃, and BiFeO₃;

the Bi-containing halide may be at least one selected from the group consisting of BiF₃, BiCl₃, BiBr₃, BiI₃, and BiAt₃; and

the solvent may be at least one selected from the group consisting of methanol, formaldehyde, ethanol, acetaldehyde, ethylene glycol (EG), diethylene glycol (DEG), dimethylformamide (DMF), benzyl alcohol, hydrazine hydrate, sodium borohydride (SBH), hydroiodic acid, acetone, dichloromethane (DCM), and trichloromethane (TCM).

The Bi-containing oxycompound may include any one selected from the group consisting of a Bi-containing oxysalt and a Bi-containing oxide.

Optionally, in step a1), the Bi-containing oxysalt may be at least one selected from the group consisting of Bi₂(SO₄)₃, Bi(NO₃)₃.5H₂O, BiPO₄, BiH(PO₃)₂, BiH₂PO₃, (BiO)₂CO₃.½H₂O, Bi₂(SO₄)₃, and BiFeO₃.

Optionally, a concentration of the Bi-containing oxycompound in the mixture of the Bi-containing oxycompound, the Bi-containing halide, and the solvent may be 1,000 g/L to 10 g/L.

Optionally, a concentration of the Bi-containing halide in the mixture of the Bi-containing oxycompound, the Bi-containing halide, and the solvent may be 1,000 g/L to 10 g/L.

Optionally, in step a1), the solvothermal reaction may be conducted at 80° C. to 180° C. for 6 h to 48 h.

Optionally, in step a2), the reduction treatment may be a UV light treatment or a reducing agent treatment.

Optionally, the UV light treatment may be conducted at 10 W to 500 W for 2 h to 12 h. Specifically, an upper limit of the intensity of UV light may be 100 W, 200 W, 300 W, or 500 W; and a lower limit of the intensity of UV light may be 10 W, 100 W, 200 W, or 300 W.

An upper limit of the irradiation time may be 4 h, 6 h, 8 h, 10 h, or 12 h; and a lower limit of the irradiation time may be 2 h, 4 h, 6 h, 8 h, or 10 h.

Optionally, the reducing agent treatment may be a calcination in the presence of a reducing agent at 300° C. to 400° C. for 2 h to 3 h.

Optionally, the reducing agent may be at least one selected from the group consisting of SBH, potassium borohydride (KBH), stannous chloride, oxalic acid, and dithizone.

Optionally, a mass ratio of the reducing agent to the oxygen vacancy-containing BiOX particle I may be 1:(100-1).

Optionally, in step a1), a mass ratio of the Bi-containing oxysalt to the Bi-containing halide may be (10-1):(0.1-1).

Optionally, in step a2), a concentration of the oxygen vacancy-containing BiOX particle II in the dispersion II of the oxygen vacancy-containing BiOX particle II may be 5,000 g/L to 100 g/L.

Optionally, in step b), the reaction may be conducted at 20° C. to 35° C. under stirring.

According to another aspect of the present disclosure, a use of the nanocomposite described above and/or a nanocomposite prepared by the preparation method of the nanocomposite described above in the preparation of a nanomaterial for PTT of a tumor is provided.

According to another aspect of the present disclosure, a use of the nanocomposite described above and/or a nanocomposite prepared by the preparation method of the nanocomposite described above in the preparation of a nanomaterial for PDT of a tumor is provided.

According to another aspect of the present disclosure, a use of the nanocomposite described above and/or a nanocomposite prepared by the preparation method of the nanocomposite described above in the preparation of a tumor-targeted drug is provided.

According to another aspect of the present disclosure, a use of the nanocomposite described above and/or a nanocomposite prepared by the preparation method of the nanocomposite described above in the preparation of a material for tumor diagnosis is provided.

According to another aspect of the present disclosure, a use of the nanocomposite described above and/or a nanocomposite prepared by the preparation method of the nanocomposite described above in the preparation of a material for tumor diagnosis in vitro and in vivo is provided.

According to another aspect of the present disclosure, a use of the nanocomposite described above and/or a nanocomposite prepared by the preparation method of the nanocomposite described above in cell isolation is provided.

According to another aspect of the present disclosure, a use of the nanocomposite described above and/or a nanocomposite prepared by the preparation method of the nanocomposite described above in a drug carrier is provided.

According to another aspect of the present disclosure, a use of the nanocomposite described above and/or a nanocomposite prepared by the preparation method of the nanocomposite described above in the preparation of a material for heavy-ion therapy is provided.

According to another aspect of the present disclosure, a use of the nanocomposite described above and/or a nanocomposite prepared by the preparation method of the nanocomposite described above in the preparation of a material for isotope diagnosis and treatment is provided.

According to another aspect of the present disclosure, a use of the nanocomposite described above and/or a nanocomposite prepared by the preparation method of the nanocomposite described above in the preparation of a material for integrated tumor diagnosis and treatment is provided.

According to another aspect of the present disclosure, a product is provided, including the nanocomposite described above and/or a nanocomposite prepared by the preparation method of the nanocomposite described above.

According to another aspect of the present disclosure, a use of the nanocomposite described above and/or a nanocomposite prepared by the preparation method of the nanocomposite described above in heavy-ion therapy is provided.

According to another aspect of the present disclosure, a use of the nanocomposite described above and/or a nanocomposite prepared by the preparation method of the nanocomposite described above in isotope diagnosis and treatment is provided.

Possible beneficial effects of the present disclosure:

1) The nanocomposite provided by the present disclosure is an oxygen vacancy-containing BiOX nanomaterial, and a band gap thereof is shortened due to the defect energy level, such that the nanocomposite can achieve a full-spectrum absorption effect. The nanocomposite can absorb NIR-II laser (1,060 nm) while retaining its photodynamic performance. The nanocomposite can be used for the combined photothermal and photodynamic therapy of tumors due to its excellent photothermal conversion performance and photodynamic performance and can also be used for CT/PAI dual-modality imaging, which can achieve the purpose of integrated tumor diagnosis and treatment.

2) With the preparation method of the nanocomposite provided by the present disclosure, a series of surface oxygen vacancy-containing 2D nanomaterials with excellent tumor diagnosis and treatment performance can be obtained through hydrothermal and solvothermal processes.

Specifically, a 2D bismuth oxyhalide crystal with an oxygen vacancy is synthesized by a solvothermal process. The bismuth oxyhalide crystal is subjected to surface modification with a polymer to obtain a safe and non-toxic photothermal reagent with small particle size, concentrated particle size distribution, high stability, prominent dispersibility in water, excellent crystallinity, excellent biocompatibility, high thermal conversion efficiency, prominent photodynamic performance, and excellent CT/PAI imaging performance.

3) The nanocomposite of the present disclosure has a CT/PAI imaging function and an imaging signal thereof is strong, such that a diagnosis and treatment reagent with excellent biocompatibility for PTT and CT/PAI imaging is finally obtained.

4) The preparation method of the nanocomposite provided by the present disclosure has the advantages of environmental friendliness, safety, reliability, simple process, low cost, high yield, easy quality control, and easy large-scale production.

5) The nanocomposite provided by the present disclosure has the characteristics of environmental friendliness, safety, simple process, low cost, and high yield. When used in the clinical diagnosis and treatment of major diseases such as tumors, the nanocomposite can improve the diagnosis and treatment of the major diseases such as tumors, thereby significantly reducing the costs of medical testing and treatment and promoting longevity and health of people.

A particle size of the 2D nanocomposite can be effectively controlled by controlling a particle size of the polymer microspheres, such that the nanocomposite can passively target different organs (for example, with a particle size of less than 10 nm, the nanocomposite can pass through the blood-brain barrier (BBB) and enter the brain; with a particle size of 10 nm to 30 nm, the nanocomposite can stay in the blood for a long time to serve as a blood-pool contrast medium (BPCM); with a particle size of 30 nm to 150 nm, the nanocomposite can enter the heart, liver, spleen, kidney, and the like through blood vessels; with a particle size of 150 nm to 250 nm, the nanocomposite can be phagocytosed by reticuloendothelial cells in the liver; and with a particle size of larger than 1 the nanocomposite can be trapped by pulmonary blood vessels).

6) The nanocomposite provided by the present disclosure can be used in the preparation of a contrast material for CT/PAI imaging, a tumor-targeted drug, a material for tumor diagnosis, and/or a drug carrier, and can also be used for in vitro tumor diagnosis, cell isolation, and the like.

7) Compared with the iodine complex preparation (a CT contrast agent used clinically), the 2D nanomaterial and/or the 2D bismuth oxyhalide nanomaterial with an oxygen vacancy of the present disclosure, when used as a CT contrast agent, shows higher contrast performance and much lower biosensitization. Therefore, the nanomaterial of the present disclosure is a very excellent CT contrast material.

8) The nanocomposite provided by the present disclosure includes bismuth, iodine, and astatine elements, which can play a role of sensibilization in cancer radiotherapy

### BRIEF DESCRIPTION OF THE DRAWINGS

FIG. 1 is a schematic diagram of a main preparation process of Example 1.

FIGS. 2A-2C show the transmission electron microscopy (TEM) images of the 2D bismuth oxyhalide nanomaterials with different numbers of oxygen vacancies obtained in Example 1, where FIG. 2A shows a white bismuth oxyhalide material without oxygen vacancies corresponding to sample 1-1; FIG. 2B shows a grey bismuth oxyhalide material with a small number of oxygen vacancies corresponding to sample 1-2; and FIG. 2C shows a black bismuth oxyhalide nanomaterial with a large number of oxygen vacancies corresponding to sample 1-3.

FIGS. 3A-3F show the theoretical calculation results of crystal structures of the 2D bismuth oxyhalide nanomaterials with different numbers of oxygen vacancies obtained in Example 1, where FIG. 3A shows a {001} crystal plane with no oxygen vacancies; FIG. 3B shows a {001} crystal plane with a small number of oxygen vacancies; FIG. 3C shows a {001} crystal plane with a large number of oxygen vacancies; FIG. 3D shows a {100} crystal plane with no oxygen vacancies; FIG. 3E shows a {100} crystal plane with a small number of oxygen vacancies; and FIG. 3F shows a {100} crystal plane with a large number of oxygen vacancies.

FIG. 4 shows the electron spin resonance (ESR)/electron paramagnetic resonance (EPR) test results of the 2D bismuth oxyhalide nanomaterials with different numbers of oxygen vacancies obtained in Example 1.

FIGS. 5A-5B show the photothermal heating curves of the 2D bismuth oxyhalide nanomaterials with different numbers of oxygen vacancies obtained in Example 1, where FIG. 5A shows the photothermal heating curves of the bismuth oxyhalide nanomaterial with a small number of oxygen vacancies and FIG. 5B shows the photothermal heating curves of the bismuth oxyhalide nanomaterial with a large number of oxygen vacancies.

FIGS. 6A-6B show the DPBF degradation rate test results of the 2D bismuth oxyhalide nanomaterials with different numbers of oxygen vacancies obtained in Example 1, where FIG. 6A shows the DPBF degradation rates of the bismuth oxyhalide nanomaterial with a small number of oxygen vacancies and FIG. 6B shows the DPBF degradation rates of the bismuth oxyhalide nanomaterial with a large number of oxygen vacancies.

FIG. 7 shows the comparison of in vivo CT imaging of the 2D bismuth oxyhalide nanomaterial with a large number of oxygen vacancies obtained in Example 1.

FIGS. 8A-8B show the X-ray photoelectron spectroscopy (XPS) test results of the 2D bismuth oxyhalide nanomaterials with different numbers of oxygen vacancies obtained in Example 1, where FIG. 8A shows XPS spectra of sample 1-2 and FIG. 8B shows XPS spectra of sample 1-3.

FIG. 9 shows the cytotoxicity test results of the 2D bismuth oxyhalide nanomaterials with different numbers of oxygen vacancies obtained in Example 1.

FIG. 10 shows the cell therapy test results of the 2D bismuth oxyhalide nanomaterial with a large number of oxygen vacancies obtained in Example 1.

### DETAILED DESCRIPTION OF THE EMBODIMENTS

The present disclosure will be described in detail below with reference to the examples, but the present disclosure is not limited to these examples.

Unless otherwise specified, the raw materials in the examples of the present disclosure are all purchased from commercial sources.

Terms

As used herein, the terms “2D nanoparticle”, “nanoparticle”, and “bismuth oxyhalide with an oxygen vacancy” can be used interchangeably, and all refer to a nanoparticle with the following characteristics:

1) the 2D nanomaterial is an oxyhalide of bismuth;

2) the 2D nanomaterial has a particle size of greater than or equal to 0.1 nm;

3) the 2D nanomaterial has a photothermal conversion efficiency of greater than or equal to 10%; and

4) the 2D bismuth oxyhalide nanomaterial with an oxygen vacancy has a CT signal intensity (grey value) of greater than or equal to 200 (SIEMENS SOMATOM Definition AS+).

As used herein, the terms “2D bismuth oxyhalide nanomaterial with an oxygen vacancy”, “nanocomposite”, and “composite nanoparticle” can be used interchangeably, and all refer to a composite obtained by coating an outer surface of a 2D nanomaterial with a nanosphere or macromolecule.

As used herein, the term “PEG” is an abbreviation for polyethylene glycol.

As used herein, the term “DEG” is an abbreviation for diethylene glycol.

As used herein, the term “PEI” is an abbreviation for polyetherimide.

As used herein, the term “PVP” is an abbreviation for polyvinylpyrrolidone.

As used herein, the term “PLGA” is an abbreviation for poly(lactic acid-glycolic acid) copolymer. As used herein, the term “CT” is an abbreviation for computed tomography.

As used herein, the term “PAI” is an abbreviation for photoacoustic imaging.

As used herein, the term “DPBF” is an abbreviation for 1,3-diphenylisobenzofuran.

As used herein, the term “room temperature” refers to 0° C. to 30° C. and preferably 4° C. to 25° C.

2D Nanomaterials in the Present Disclosure

The BiOX (X=F, Cl, Br, I, or At) nanomaterials are excellent semiconductor photocatalysts. With special electronic structures, the BiOX nanomaterials have been reported to have a strong laser-induced ROS production ability, and Bi 6s and O 2p can form a prominent hybrid valence band. The hybridization of Bi 6s and O 2p makes the valence band dispersed to a large extent, which is conducive to the migration of light-induced holes in the valence band and the progress of an oxidation reaction. Therefore, ultrathin nanosheets of such nanomaterials have received more and more attention in energy conversion and storage. Such an ultrathin nanosheet with a 2D structure makes photoexcited EHPs reach a surface more easily than EHPs generated in vivo, which reduces the recombination chance. The atomic thickness and surface distortion and defect of an ultrathin 2D crystal play an important role in the electronic structural modification and performance improvement of the crystal.

However, most of the BiOX nanomaterials have a wide band gap, can only be excited by high-energy UV light or X-rays, and are only used in the radiotherapy of tumors, which inevitably causes damage to healthy tissues. Moreover, due to the wide band gap, BiOX nanomaterials do not possess photothermal properties. Inspired by the above analysis, the present disclosure proposes an ultrathin BiOX nanosheet with a large number of surface/subsurface defects, which introduces the photothermal properties while retaining the photoexcited ROS production ability to enable the efficient diagnosis and treatment of tumors.

In the present disclosure, a surface of the 2D nanomaterial is coated with a polymer microsphere to significantly enhance the biocompatibility of the 2D nanomaterial and reduce the toxicity of the nanomaterial (especially when it is used at a high dosage).

General Test Methods

TEM

Test instrument: JEOL-2100 transmission electron microscope; test conditions: 200 Kv and 101 μA; and nanoparticles to be tested are first dispersed in water and then tested.

CT Value Measurement

Test instrument: SIEMENS SOMATOM Definition AS+; and test conditions may include tube voltage of 80 kV and tube current of 150 mAs.

Small Animal CT Imaging

Test instrument: SIEMENS SOMATOM Definition AS+; and test conditions may include tube voltage of 80 kV, 100 kv, and 120 kv and tube current of 150 mAs.

DPBF Degradation Experiment

10 mL of an ethanol solution with DPBF at a concentration of 50 mg/mL is mixed with a 100 μg/mL oxygen vacancy-containing bismuth oxychloride material solution. The resulting mixture is irradiated for 1 h at a laser power density of 50 mW cm⁻². 1 mL of a sample is taken at different time points. The absorbance of a supernatant of the sample at 400 nm is determined by ultraviolet-visible (UV-Vis) spectrophotometry.

Cytotoxicity Test

1. 4T1 cells are prepared into a suspension with a concentration of 1*10\hat{ }6/mL. 100 μL of the suspension is taken and dispersed in 100 μL of a medium composed of 95 v/v % 1640 medium and 5 v/v % fetal bovine serum (FBS). The resulting dispersion is added to a 96-well plate and incubated overnight.

2. The medium is removed, then 100 μL of each of the nanocomposites in Example 1 is added to each well at different concentrations of 100 m/mL, 200 m/mL, 300 m/mL, 400 μg/mL, and 500 m/mL, and the cells are further incubated for 24 h.

3. 20 h later, the nanocomposites are each taken out, the plate is washed 2 to 3 times with phosphate buffered saline (PBS), then 100 μL of the above medium and 5% methyl thiazolyl tetrazolium (MTT) (dissolved in dimethyl sulfoxide (DMSO)) are added, and then the cells are further incubated for 4 h.

4. The liquid in each well is removed, 100 μL of DMSO is added, the absorbance of each well of the 96-well plate at 550 nm is determined by a microplate reader, and a cell viability is calculated.

Cell Therapy Test

1. 4T1 cells are prepared into a suspension with a concentration of 1*10\hat{ }6/mL. 100 μL of the suspension is taken and dispersed in 100 μL of a medium composed of 95 v/v % 1640 medium and 5 v/v % FBS. The resulting dispersion is added to a 96-well plate and incubated overnight.

2. The medium is removed, then 100 μL of each of the nanocomposites in Example 1 is added to each well at different concentrations of 100m/mL, 200m/mL, 300m/mL, 400 μg/mL, and 500m/mL, and the cells are further incubated for 4 h.

3. 4 h later, the nanocomposites are each taken out, the plate is washed 2 to 3 times with PBS, and then 100 μL of the above medium is added.

4. Each well of the 96-well plate is irradiated with an 808 nm laser at a laser power density of 1.0 W/cm².

5. After the irradiation, the cells are further incubated for 20 h. Then 5% MTT (dissolved in DMSO) is added, and the cells are further incubated for 4 h.

6. The liquid in each well is removed, 100 μL of DMSO is added, the absorbance of each well of the 96-well plate at 550 nm is determined by a microplate reader, and a cell viability is calculated.

### Example 1

Preparation of a White Bismuth Oxyhalide Material without Oxygen Vacancies

(1-1-1) 486 mg of Bi(NO₃)₃.5H₂O, 400 mg of PVP, and 455 mg of mannitol were weighed, mixed, and dissolved in 25 mL of ultrapure water (UPW), and a resulting mixed solution was stirred for 10 min and then subjected to an ultrasonic treatment for 10 min in an ultrasonic machine to obtain a solution a;

(1-1-2) Under continuous stirring, 5 mL of a saturated NaCl solution was slowly added dropwise to the solution a through a syringe to obtain a white homogeneous suspension b;

(1-1-3) The white homogeneous suspension b was subjected to ultrasonic dispersion for 10 min and then transferred to a 50 ml polytetrafluoroethylene (PTFE) hydrothermal reactor to undergo a hydrothermal reaction at 160° C. for 3 h. A resulting reaction solution was cooled. A resulting precipitate was separated, washed 8 times alternately with water and ethanol, and then dried to obtain the white bismuth oxyhalide material c for later use, which was denoted as sample 1-1.

Preparation of a Grey Bismuth Oxyhalide Material with a Small Number of Oxygen Vacancies and a Black Bismuth Oxyhalide Nanomaterial with a Large Number of Oxygen Vacancies

(1-2-1) 0.97 g of Bi(NO₃)₃.5H₂O and 0.315 g of BiCl₃ were weighed and dissolved in 40 mL of DEG. A resulting mixed solution was continuously stirred for 10 min on a magnetic stirrer at 80 r/min and then subjected to an ultrasonic treatment for 10 min by an ultrasonic machine to obtain a solution d.

(1-2-2) The solution d was transferred to a PTFE-lined hydrothermal reactor to undergo a reaction at 180° C. for 12 h; and

after the reaction was completed, a resulting reaction solution was cooled to room temperature, a resulting supernatant was removed, and a resulting precipitate was washed 4 times with water and ethanol, and then dried to obtain a grey bismuth oxyhalide material e with a small number of oxygen vacancies for later use, which was denoted as sample 1-2.

(1-2-3) 50 mg of the grey bismuth oxyhalide material e with a small number of oxygen vacancies was taken and dispersed in 10 mL of an aqueous solution. A resulting dispersion was transferred to a photochemical reactor and irradiated under a 500 W mercury lamp for 2 h to obtain a black bismuth oxyhalide nanomaterial-dispersed system f.

(1-2-4) The black bismuth oxyhalide nanomaterial-dispersed system f was centrifuged at 10,000 r/min, and a resulting precipitate was washed 4 times with water and ethanol to obtain a solid, which was denoted as sample 1-3. The solid was dispersed in 10 mL of ethanol to obtain a dispersion.

(1-2-5) 10 mL of a solution of PEG in ethanol (with a concentration of 50 mg/mL) was added to the dispersion obtained in step (1-2-4), and a resulting mixed solution was continuously stirred for 24 h on a magnetic stirrer at 80 r/min. After a reaction was completed, a resulting mixture was centrifuged at 10,000 r/min. A resulting precipitate was washed 4 times with water and ethanol to obtain a black 2D bismuth oxyhalide nanomaterial with a large number of oxygen vacancies, which was denoted as sample 1-4 and stored at 4° C.

Results

The 2D bismuth oxyhalide nanomaterials with different numbers of oxygen vacancies obtained in Example 1 were each subjected to TEM, ESR/EPR, and photothermal heating tests (for material characterization), cytotoxicity and cell therapy tests, animal therapy and animal tissue section tests (for in vivo toxicity analysis), and CT value measurement and CT imaging performance tests.

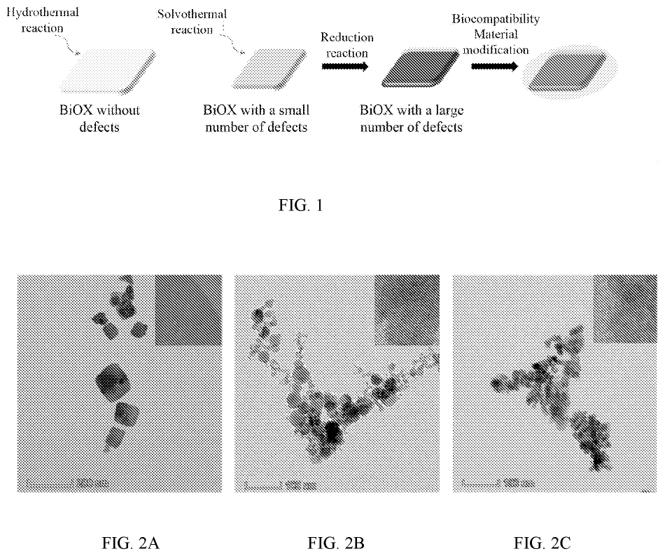

FIG. 1 shows a preparation process and technical route of the material.

It can be known from FIG. 1 that core technologies of the present disclosure are mainly as follows: a hydrothermal reaction is used to synthesize a defect-free 2D bismuth oxyhalide material; a solvothermal reaction is used to synthesize a 2D bismuth oxyhalide material with a small number of oxygen vacancies; and the UV light reduction is used to produce more oxygen vacancies to reduce a band gap of the material, such that the material can achieve full-spectrum absorption and can achieve PTT and PDT simultaneously under NIR irradiation. The bismuth element enables CT/PAI dual-modality imaging, and thus the bismuth oxyhalide material can achieve the combined photothermal and photodynamic therapy of a tumor under the accurate guidance of dual-modality imaging.

FIGS. 2A-2C show the TEM images of the 2D bismuth oxyhalide nanomaterials with different numbers of oxygen vacancies obtained in Example 1, where FIG. 2A shows a white bismuth oxyhalide material without oxygen vacancies corresponding to sample 1-1; FIG. 2B shows a grey bismuth oxyhalide material with a small number of oxygen vacancies corresponding to sample 1-2; and FIG. 2C shows a black bismuth oxyhalide nanomaterial with a large number of oxygen vacancies corresponding to sample 1-3. It can be seen from FIGS. 2A-2C that the synthesized materials are BiOCl materials.

It can be known from FIGS. 2A-2C that the 2D bismuth oxyhalide nanomaterials with different numbers of oxygen vacancies have an average particle size of about 100 nm, and lattice fringes are obvious in the high-resolution TEM images.

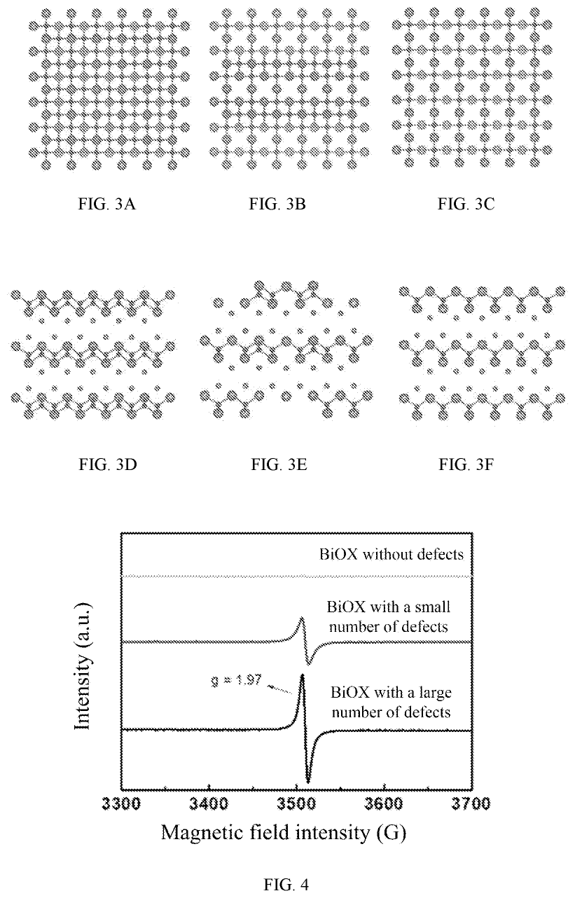

FIGS. 3A-3F show the theoretical calculation results of crystal structures of the 2D bismuth oxyhalide nanomaterials with different numbers of oxygen vacancies obtained in Example 1, where FIG. 3A shows a {001} crystal plane with no oxygen vacancies; FIG. 3B shows a {001} crystal plane with a small number of oxygen vacancies; FIG. 3C shows a {001} crystal plane with a large number of oxygen vacancies; FIG. 3D shows a {100} crystal plane with no oxygen vacancies; FIG. 3E shows a {100} crystal plane with a small number of oxygen vacancies; and FIG. 3F shows a {100} crystal plane with a large number of oxygen vacancies.

It can be seen from FIGS. 3A-3F that, among the 2D bismuth oxyhalide nanomaterials with different numbers of oxygen vacancies, the crystal structure constantly varies with the increase of oxygen vacancies.

FIG. 4 shows the ESR/EPR test results of the 2D bismuth oxyhalide nanomaterials with different numbers of oxygen vacancies obtained in Example 1, where defect-free BiOX corresponds to sample 1-1, BiOX with a small number of defects corresponds to sample 1-2, and BiOX with a large number of defects corresponds to sample 1-3.

It can be seen from FIG. 4 that, among the 2D bismuth oxyhalide nanomaterials with different numbers of oxygen vacancies, the oxygen vacancy peak in the ESR test result constantly increases with the increase of oxygen vacancies.

The 2D bismuth oxyhalide nanomaterials with different numbers of oxygen vacancies prepared in Example 1 were each subjected to an XPS test using an instrument of Axis Ultra DLD X-ray photoelectron spectrometer. The conventional XPS qualitative, semi-quantitative, valence band, and chemical valence analysis were conducted with an analysis element of oxygen (O).

FIGS. 8A-8B show the XPS test results of the 2D bismuth oxyhalide nanomaterials with different numbers of oxygen vacancies obtained in Example 1, where FIG. 8A shows XPS spectra of sample 1-2 and FIG. 8B shows XPS spectra of sample 1-3. In FIGS. 8A-8B, “CPS” represents a synthesis peak, “abs” represents adsorbed oxygen, “O—H” represents an oxygen vacancy, and “O—Bi” represents a bismuth-oxygen bond. It can be seen from (a) in FIGS. 8A-8B that a proportion of oxygen vacancies in sample 1-2 is 30%. It can be seen from (b) in FIGS. 8A-8B that a proportion of oxygen vacancies in sample 1-3 is about 50%.

The 2D bismuth oxyhalide nanomaterials with different numbers of oxygen vacancies prepared in Example 1 were each subjected to a photothermal test, and a test process was as follows: the materials (dispersed in water) were each placed in a cuvette at different concentrations (100 μg/mL, 200 μg/mL, 300 μg/mL, 400 μg/mL, and 500 μg/mL) and then irradiated with a 1,060 nm NIR laser (at a power density of 1 W/cm²), and the temperature changes of the materials were measured with a thermal imager.

FIGS. 5A-5B show the photothermal heating data of the 2D bismuth oxyhalide nanomaterials with different numbers of oxygen vacancies obtained in Example 1, where FIG. 5A shows the photothermal heating curves of the 2D bismuth oxyhalide nanomaterial with a small number of oxygen vacancies (sample 1-2) and FIG. 5B shows the photothermal heating curves of the 2D bismuth oxyhalide nanomaterial with a large number of oxygen vacancies (sample 1-3).

It can be seen from FIG. 5A that the 2D bismuth oxyhalide nanomaterial with a small number of oxygen vacancies exhibits a poor photothermal heating effect under 1,060 nm laser; and it can be seen from FIG. 5B that the 2D bismuth oxyhalide nanomaterial with a large number of oxygen vacancies exhibits a prominent photothermal heating effect under 1,060 nm laser and has a high photothermal conversion efficiency.

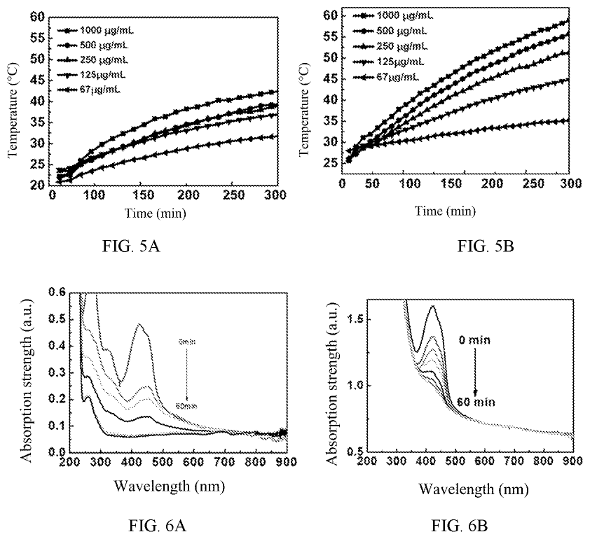

FIGS. 6A-6B show the in vitro DPBF degradation test data of the bismuth oxyhalide nanomaterials with different numbers of oxygen vacancies obtained in Example 1, where FIG. 6A shows the DPBF degradation test data of the 2D bismuth oxyhalide nanomaterial with a small number of oxygen vacancies (sample 1-2) and FIG. 6B shows the DPBF degradation test data of the 2D bismuth oxyhalide nanomaterial with a large number of oxygen vacancies (sample 1-3).

It can be seen from FIGS. 6A-6B that the 2D bismuth oxyhalide nanomaterials with different numbers of oxygen vacancies all exhibit a strong ROS production ability.

The 2D bismuth oxyhalide nanomaterials with different numbers of oxygen vacancies prepared in Example 1 were each subjected to a CT imaging test, and a test process was as follows: the bismuth oxyhalide materials in different concentrations and the CT contrast agent iopamidol used clinically were each dispersed in 5% agar at the same molar concentration, fixed, and tested by SIEMENS SOMATOM Definition AS for CT value and CT imaging under a tube voltage of 80 kV and tube current of 150 mAs.

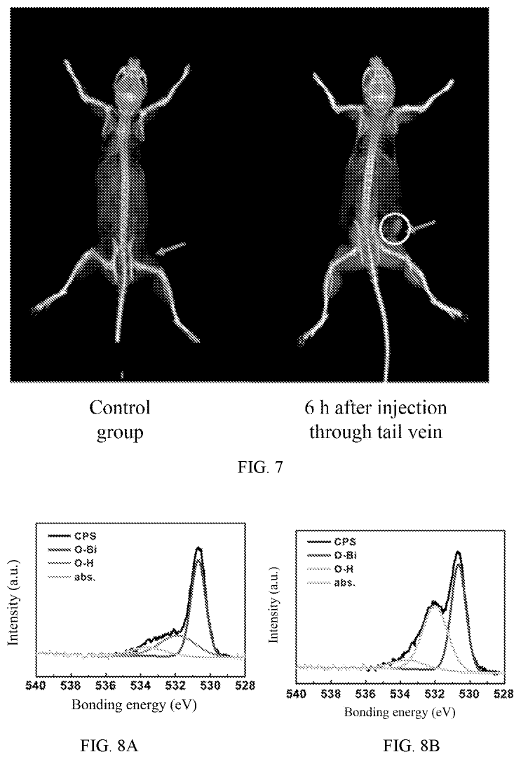

FIG. 7 shows the in vivo CT imaging results of the 2D bismuth oxyhalide nanomaterials with different numbers of oxygen vacancies obtained in Example 1. The right panel shows the CT imaging of the bismuth oxyhalide material with a large number of oxygen vacancies in a mouse injected with the bismuth oxyhalide material through the tail vein. The left panel shows the CT imaging of a control group injected with the same amount of an injection without the bismuth oxyhalide material. A test process was as follows: The mouse was injected with 100 μL of an aqueous solution of sample 1-3 (the bismuth oxyhalide nanomaterial with a large number of oxygen vacancies) at a concentration of 3 mg/mL, and 6 h later, the CT imaging test was conducted. The imaging result of the experimental group is shown in the right panel of FIG. 7, and the imaging result of the control group is shown in the left panel of FIG. 7.

It can be seen from FIG. 7 that the 2D bismuth oxyhalide nanomaterial with a large number of oxygen vacancies obtained in Example 1 has a strong signal intensity, and can be enriched in a tumor area through the EPR effect after a period of time, indicating that the 2D bismuth oxyhalide nanomaterial with a large number of oxygen vacancies obtained in Example 1 is a prominent CT imaging material.

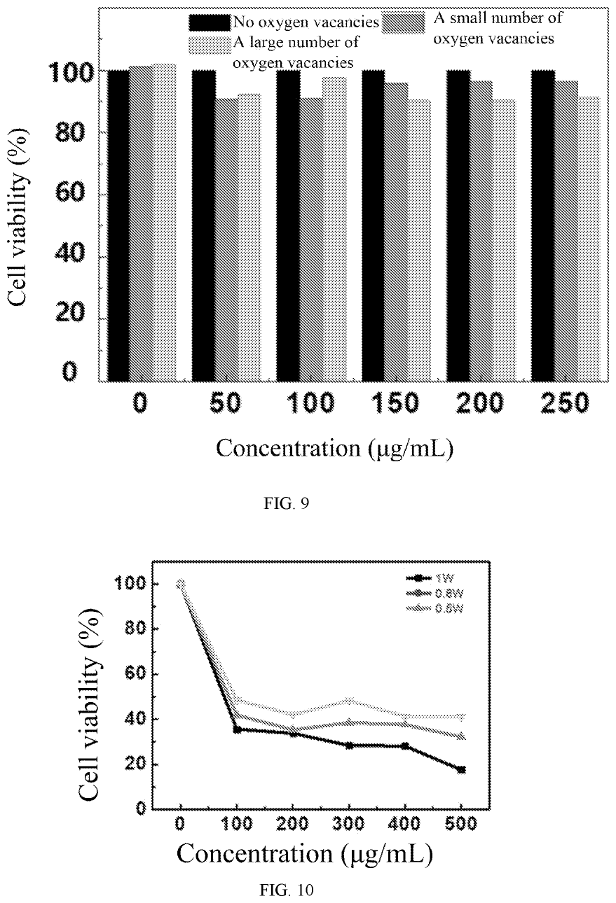

The 2D bismuth oxyhalide nanomaterials with different numbers of oxygen vacancies prepared in Example 1 were each subjected to a cytotoxicity test, and test results are shown in FIG. 9. In FIG. 9, “no oxygen vacancies” corresponds to sample 1-1, a nanocomposite prepared according to steps (1-2-3) and (1-2-4); “a small number of oxygen vacancies” corresponds to sample 1-2, a nanocomposite prepared according to steps (1-2-3) and (1-2-4); and “a large number of oxygen vacancies” corresponds to sample 1-4. The test results show that, in the presence of the different nanocomposites at different concentrations, a cell viability is nearly 100%, indicating that the nanocomposites have little toxicity to cells.

The 2D bismuth oxyhalide nanomaterial with a large number of oxygen vacancies (sample 1-4) prepared in Example 1 was subjected to a cell therapy test, and test results are shown in FIG. 10. The test results show that, under IR irradiation at different powers, different concentrations of the nanocomposite lead to a prominent killing effect on cancer cells, and nearly 50% of the cancer cells are killed.

### Example 2

Preparation of a Grey Bismuth Oxyhalide Material with a Small Number of Oxygen Vacancies and a Black Bismuth Oxyhalide Nanomaterial with a Large Number of Oxygen Vacancies

(2-2-1) 0.97 g of Bi(NO₃)₃.5H₂O and 0.315 g of BiCl₃ were weighed and dissolved in 40 mL of DEG. A resulting mixed solution was continuously stirred for 10 min on a magnetic stirrer at 80 r/min and then subjected to an ultrasonic treatment for 10 min in an ultrasonic machine to obtain a solution d.

(2-2-2) The solution d was transferred to a PTFE-lined hydrothermal reactor to undergo a reaction at 160° C. for 12 h; and

after the reaction was completed, a resulting reaction solution was cooled to room temperature, a resulting supernatant was removed, and a resulting precipitate was washed 4 times with water and ethanol, and then dried to obtain a grey bismuth oxyhalide material e with a small number of oxygen vacancies for later use, which was denoted as sample 2-2.

(2-2-3) 50 mg of the grey bismuth oxyhalide material e with a small number of oxygen vacancies was taken and dispersed in 10 mL of an aqueous solution. A resulting dispersion was transferred to a photochemical reactor and irradiated under a 500 W mercury lamp for 2 h to obtain a black bismuth oxyhalide nanomaterial-dispersed system f.

(2-2-4) The black bismuth oxyhalide nanomaterial-dispersed system f was centrifuged at 10,000 r/min. A resulting precipitate was washed 4 times with water and ethanol to obtain a solid, which was denoted as sample 2-3. The solid was dispersed in 10 mL of ethanol to obtain a dispersion.

(2-2-5) 10 mL of a solution of PEG in ethanol (with a concentration of 50 mg/mL) was added to the dispersion obtained in step (2-2-4). A resulting mixed solution was continuously stirred for 24 h on a magnetic stirrer at 80 r/min. After a reaction was completed, a resulting mixture was centrifuged at 10,000 r/min. A resulting precipitate was washed 4 times with water and ethanol to obtain a black 2D bismuth oxyhalide nanomaterial with a large number of oxygen vacancies, which was denoted as sample 2-4, and stored at 4° C.

Results

The TEM, DPBF degradation rate, photothermal conversion efficiency, cytotoxicity, CT value measurement, and in vivo CT imaging test results of the 2D bismuth oxyhalide composites with different numbers of oxygen vacancies obtained in Example 2 are basically the same as those of Example 1. XPS shows that a proportion of oxygen vacancies in the grey bismuth oxyhalide nanomaterial is 20% to 40% and a proportion of oxygen vacancies in the black bismuth oxyhalide nanomaterial is greater than 40%.

### Example 3

Preparation of a Grey Bismuth Oxyhalide Material with a Small Number of Oxygen Vacancies and a Black Bismuth Oxyhalide Nanomaterial with a Large Number of Oxygen Vacancies

(3-2-1) 0.97 g of Bi(NO₃)₃.5H₂O and 0.315 g of BiCl₃ were weighed and dissolved in 40 mL of DEG. A resulting mixed solution was continuously stirred for 10 min on a magnetic stirrer at 80 r/min and then subjected to an ultrasonic treatment for 10 min in an ultrasonic machine to obtain a solution d.

(3-2-2) The solution d was transferred to a PTFE-lined hydrothermal reactor to undergo a reaction at 140° C. for 12 h; and

after the reaction was completed, a resulting reaction solution was cooled to room temperature, a resulting supernatant was removed, and a resulting precipitate was washed 4 times with water and ethanol, and then dried to obtain a grey bismuth oxyhalide material e with a small number of oxygen vacancies for later use, which was denoted as sample 3-2.

(3-2-3) 50 mg of the grey bismuth oxyhalide material e with a small number of oxygen vacancies was taken and dispersed in 10 mL of an aqueous solution. A resulting dispersion was transferred to a photochemical reactor and irradiated under a 500 W mercury lamp for 2 h to obtain a black bismuth oxyhalide nanomaterial-dispersed system f.

(3-2-4) The black bismuth oxyhalide nanomaterial-dispersed system f was centrifuged at 10,000 r/min. A resulting precipitate was washed 4 times with water and ethanol to obtain a solid, which was denoted as sample 3-3. The solid was dispersed in 10 mL of ethanol to obtain a dispersion.

(3-2-5) 10 mL of a solution of PEG in ethanol (with a concentration of 50 mg/mL) was added to the dispersion obtained in step (3-2-4). A resulting mixed solution was continuously stirred for 24 h on a magnetic stirrer at 80 r/min. After a reaction was completed, a resulting mixture was centrifuged at 10,000 r/min. A resulting precipitate was washed 4 times with water and ethanol to obtain a black 2D bismuth oxyhalide nanomaterial with a large number of oxygen vacancies, which was denoted as sample 3-4, and stored at 4° C.

Results

The TEM, DPBF degradation rate, photothermal conversion efficiency, cytotoxicity, CT value measurement, and in vivo CT imaging test results of the 2D bismuth oxyhalide composites with different numbers of oxygen vacancies obtained in Example 3 are basically the same as those of Example 1. XPS shows that a proportion of oxygen vacancies in the grey bismuth oxyhalide nanomaterial is 20% to 40% and a proportion of oxygen vacancies in the black bismuth oxyhalide nanomaterial is greater than 40%.

### Example 4

Preparation of a Grey Bismuth Oxyhalide Material with a Small Number of Oxygen Vacancies and a Black Bismuth Oxyhalide Nanomaterial with a Large Number of Oxygen Vacancies

(4-2-1) 0.97 g of Bi(NO₃)₃.5H₂O and 0.315 g of BiCl₃ were weighed and dissolved in 40 mL of DEG. A resulting mixed solution was continuously stirred for 10 min on a magnetic stirrer at 80 r/min and then subjected to an ultrasonic treatment for 10 min in an ultrasonic machine to obtain a solution d.

(4-2-2) The solution d was transferred to a PTFE-lined hydrothermal reactor to undergo a reaction at 140° C. for 12 h; and

after the reaction was completed, a resulting reaction solution was cooled to room temperature, a resulting supernatant was removed, and a resulting precipitate was washed 4 times with water and ethanol, and then dried to obtain a grey bismuth oxyhalide material e with a small number of oxygen vacancies for later use, which was denoted as sample 4-2.

(4-2-3) 50 mg of the grey bismuth oxyhalide material e with a small number of oxygen vacancies was taken and dispersed in 10 mL of an aqueous solution. A resulting dispersion was transferred to a photochemical reactor and irradiated under a 300 W mercury lamp for 4 h to obtain a black bismuth oxyhalide nanomaterial-dispersed system f.

(4-2-4) The black bismuth oxyhalide nanomaterial-dispersed system f was centrifuged at 10,000 r/min. A resulting precipitate was washed 4 times with water and ethanol to obtain a solid, which was denoted as sample 4-3. The solid was dispersed in 10 mL of ethanol to obtain a dispersion.

(4-2-5) 10 mL of a solution of PEG in ethanol (with a concentration of 50 mg/mL) was added to the dispersion obtained in step (4-2-4). A resulting mixed solution was continuously stirred for 24 h on a magnetic stirrer at 80 r/min. After a reaction was completed, a resulting mixture was centrifuged at 10,000 r/min, and a resulting precipitate was washed 4 times with water and ethanol to obtain a black 2D bismuth oxyhalide nanomaterial with a large number of oxygen vacancies, which was denoted as sample 4-4, and stored at 4° C.

Results

The TEM, DPBF degradation rate, photothermal conversion efficiency, cytotoxicity, CT value measurement, and in vivo CT imaging test results of the 2D bismuth oxyhalide composites with different numbers of oxygen vacancies obtained in Example 4 are basically the same as those of Example 1. XPS shows that a proportion of oxygen vacancies in the grey bismuth oxyhalide nanomaterial is 20% to 40% and a proportion of oxygen vacancies in the black bismuth oxyhalide nanomaterial is greater than 40%.

### Example 5

Preparation of a Grey Bismuth Oxyhalide Material with a Small Number of Oxygen Vacancies and a Black Bismuth Oxyhalide Nanomaterial with a Large Number of Oxygen Vacancies

(5-2-1) 0.97 g of Bi(NO₃)₃.5H₂O and 0.315 g of BiCl₃ were weighed and dissolved in 40 mL of DEG. A resulting mixed solution was continuously stirred for 10 min on a magnetic stirrer at 80 r/min and then subjected to an ultrasonic treatment for 10 min in an ultrasonic machine to obtain a solution d.

(5-2-2) The solution d was transferred to a PTFE-lined hydrothermal reactor to undergo a reaction at 180° C. for 6 h; and

after the reaction was completed, a resulting reaction solution was cooled to room temperature, a resulting supernatant was removed, and a resulting precipitate was washed 4 times with water and ethanol, and then dried to obtain a grey bismuth oxyhalide material e with a small number of oxygen vacancies for later use, which was denoted as sample 5-2.

(5-2-3) 50 mg of the grey bismuth oxyhalide material e with a small number of oxygen vacancies was taken and dispersed in 10 mL of an aqueous solution. A resulting dispersion was transferred to a photochemical reactor and irradiated under a 300 W mercury lamp for 4 h to obtain a black bismuth oxyhalide nanomaterial-dispersed system f.

(5-2-4) The black bismuth oxyhalide nanomaterial-dispersed system f was centrifuged at 10,000 r/min. A resulting precipitate was washed 4 times with water and ethanol to obtain a solid, which was denoted as sample 5-3. The solid was dispersed in 10 mL of ethanol to obtain a dispersion.

(5-2-5) 10 mL of a solution of PEG in ethanol (with a concentration of 50 mg/mL) was added to the dispersion obtained in step (5-2-4). A resulting mixed solution was continuously stirred for 24 h on a magnetic stirrer at 80 r/min. After a reaction was completed, a resulting mixture was centrifuged at 10,000 r/min. A resulting precipitate was washed 4 times with water and ethanol to obtain a black 2D bismuth oxyhalide nanomaterial with a large number of oxygen vacancies, which was denoted as sample 5-4, and stored at 4° C.

The TEM, DPBF degradation rate, photothermal conversion efficiency, cytotoxicity, CT value measurement, and in vivo CT imaging test results of the 2D bismuth oxyhalide composites with different numbers of oxygen vacancies obtained in Example 5 are basically the same as those of Example 1. XPS shows that a proportion of oxygen vacancies in the grey bismuth oxyhalide nanomaterial is 20% to 40% and a proportion of oxygen vacancies in the black bismuth oxyhalide nanomaterial is greater than 40%.

### Example 6

Preparation of a Grey Bismuth Oxyhalide Material with a Small Number of Oxygen Vacancies and a Black Bismuth Oxyhalide Nanomaterial with a Large Number of Oxygen Vacancies

(6-2-1) 0.97 g of Bi(NO₃)₃.5H₂O and 0.315 g of BiCl₃ were weighed and dissolved in 40 mL of DEG. A resulting mixed solution was continuously stirred for 10 min on a magnetic stirrer at 80 r/min and then subjected to an ultrasonic treatment for 10 min in an ultrasonic machine to obtain a solution d.

(6-2-2) The solution d was transferred to a PTFE-lined hydrothermal reactor to undergo a reaction at 180° C. for 6 h; and

after the reaction was completed, a resulting reaction solution was cooled to room temperature, a resulting supernatant was removed, and a resulting precipitate was washed 4 times with water and ethanol, and then dried to obtain a grey bismuth oxyhalide material e with a small number of oxygen vacancies for later use, which was denoted as sample 5-2.

(6-2-3) 50 mg of the grey bismuth oxyhalide material e with a small number of oxygen vacancies was taken and dispersed in 10 mL of an aqueous solution. A resulting dispersion was transferred to a photochemical reactor and irradiated under a 300 W mercury lamp for 4 h to obtain a black bismuth oxyhalide nanomaterial-dispersed system f.

(6-2-4) The black bismuth oxyhalide nanomaterial-dispersed system f was centrifuged at 10,000 r/min. A resulting precipitate was washed 4 times with water and ethanol to obtain a solid, which was denoted as sample 6-3. The solid was dispersed in 10 mL of ethanol to obtain a dispersion.

(6-2-5) 10 mL of a solution of PEG in ethanol (with a concentration of 25 mg/mL) was added to the dispersion obtained in step (6-2-4). A resulting mixed solution was continuously stirred for 24 h on a magnetic stirrer at 80 r/min. After a reaction was completed, a resulting mixture was centrifuged at 10,000 r/min. A resulting precipitate was washed 4 times with water and ethanol to obtain a black 2D bismuth oxyhalide nanomaterial with a large number of oxygen vacancies, which was denoted as sample 6-4 and stored at 4° C.

Results

The TEM, DPBF degradation rate, photothermal conversion efficiency, cytotoxicity, CT value measurement, and in vivo CT imaging test results of the 2D bismuth oxyhalide composites with different numbers of oxygen vacancies obtained in Example 6 are basically the same as those of Example 1. XPS shows that a proportion of oxygen vacancies in the grey bismuth oxyhalide nanomaterial is 20% to 40% and a proportion of oxygen vacancies in the black bismuth oxyhalide nanomaterial is greater than 40%.

### Example 7

Preparation of a Grey Bismuth Oxyhalide Material with a Small Number of Oxygen Vacancies and a Black Bismuth Oxyhalide Nanomaterial with a Large Number of Oxygen Vacancies

(7-2-1) 0.97 g of Bi(NO₃)₃.5H₂O and 0.315 g of BiCl₃ were weighed and dissolved in 40 mL of DEG. A resulting mixed solution was continuously stirred for 10 min on a magnetic stirrer at 80 r/min and then subjected to an ultrasonic treatment for 10 min in an ultrasonic machine to obtain a solution d.

(7-2-2) The solution d was transferred to a PTFE-lined hydrothermal reactor to undergo a reaction at 180° C. for 6 h; and

after the reaction was completed, a resulting reaction solution was cooled to room temperature, a resulting supernatant was removed, and a resulting precipitate was washed 4 times with water and ethanol, and then dried to obtain a grey bismuth oxyhalide material e with a small number of oxygen vacancies for later use, which was denoted as sample 7-2.

(7-2-3) 50 mg of the grey bismuth oxyhalide material e with a small number of oxygen vacancies was taken and mixed with 2 g of dithizone. A resulting powdery mixture was transferred to a tube furnace and calcined at 400° C. for 2 h to obtain a bismuth oxyhalide nanomaterial d with a large number of oxygen vacancies.

(7-2-4) The bismuth oxyhalide nanomaterial d was washed 8 times with water and ethanol to obtain a solid, which was denoted as sample 7-3; the solid was dispersed in 10 mL of ethanol to obtain a dispersion.

(7-2-5) 10 mL of a solution of PEG in ethanol (with a concentration of 25 mg/mL) was added to the dispersion obtained in step (7-2-4). A resulting mixed solution was continuously stirred for 24 h on a magnetic stirrer at 80 r/min. After a reaction was completed, a resulting mixture was centrifuged at 10,000 r/min. A resulting precipitate was washed 4 times with water and ethanol to obtain a black 2D bismuth oxyhalide nanomaterial with a large number of oxygen vacancies, which was denoted as sample 7-4, and stored at 4° C.

The TEM, DPBF degradation rate, photothermal conversion efficiency, cytotoxicity, CT value measurement, and in vivo CT imaging test results of the 2D bismuth oxyhalide composites with different numbers of oxygen vacancies obtained in Example 7 are basically the same as those of Example 1. XPS shows that a proportion of oxygen vacancies in the grey bismuth oxyhalide nanomaterial is 20% to 40% and a proportion of oxygen vacancies in the black bismuth oxyhalide nanomaterial is greater than 40%.

### Example 8

Preparation of a Grey Bismuth Oxyhalide Material with a Small Number of Oxygen Vacancies and a Black Bismuth Oxyhalide Nanomaterial with a Large Number of Oxygen Vacancies

(8-2-1) 0.97 g of Bi(NO₃)₃.5H₂O and 0.315 g of BiCl₃ were weighed and dissolved in 40 mL of DEG. A resulting mixed solution was continuously stirred for 10 min on a magnetic stirrer at 80 r/min and then subjected to an ultrasonic treatment for 10 min by an ultrasonic machine to obtain a solution d.

(8-2-2) The solution d was transferred to a PTFE-lined hydrothermal reactor to undergo a reaction at 180° C. for 6 h; and

after the reaction was completed, a resulting reaction solution was cooled to room temperature, a resulting supernatant was removed, and a resulting precipitate was washed 4 times with water and ethanol, and then dried to obtain a grey bismuth oxyhalide material e with a small number of oxygen vacancies for later use, which was denoted as sample 8-2.

(8-2-3) 50 mg of the grey bismuth oxyhalide material e with a small number of oxygen vacancies was taken and dispersed in 10 mL of an aqueous solution. A resulting dispersion was transferred to a photochemical reactor and irradiated under a 500 W mercury lamp for 2 h to obtain a black bismuth oxyhalide nanomaterial-dispersed system f.

(8-2-4) The black bismuth oxyhalide nanomaterial-dispersed system f was centrifuged at 10,000 r/min. A resulting precipitate was washed 8 times with water and ethanol to obtain a solid, which was denoted as sample 8-3. The solid was dispersed in 10 mL of ethanol to obtain a dispersion.

(8-2-5) 10 mL of a solution of PEI in DMF (with a concentration of 50 mg/mL) was added to the dispersion obtained in step (8-2-4). A resulting mixed solution was continuously stirred for 24 h on a magnetic stirrer at 80 r/min. After a reaction was completed, a resulting mixture was centrifuged at 10,000 r/min. A resulting precipitate was washed 4 times with water and ethanol to obtain a black 2D bismuth oxyhalide nanomaterial with a large number of oxygen vacancies, which was denoted as sample 8-4, and stored at 4° C.

The TEM, DPBF degradation rate, photothermal conversion efficiency, cytotoxicity, CT value measurement, and in vivo CT imaging test results of the 2D bismuth oxyhalide composites with different numbers of oxygen vacancies obtained in Example 8 are basically the same as those of Example 1. XPS shows that a proportion of oxygen vacancies in the grey bismuth oxyhalide nanomaterial is 20% to 40% and a proportion of oxygen vacancies in the black bismuth oxyhalide nanomaterial is greater than 40%.

### Example 9

(9-2-1) 0.97 g of Bi(NO₃)₃.5H₂O and 0.315 g of BiCl₃ were weighed and dissolved in 40 mL of DEG. A resulting mixed solution was continuously stirred for 10 min on a magnetic stirrer at 80 r/min and then subjected to an ultrasonic treatment for 10 min in an ultrasonic machine to obtain a solution d.

(9-2-2) The solution a was transferred to a PTFE-lined hydrothermal reactor to undergo a reaction at 180° C. for 6 h.

(9-2-3) After the reaction was completed, a resulting reaction solution was cooled to room temperature, and a resulting supernatant was removed. A resulting precipitate was washed 4 times with water and ethanol and then dried to obtain a grey bismuth oxyhalide material e with a small number of oxygen vacancies for later use.

(9-2-4) 50 mg of the grey bismuth oxyhalide material e with a small number of oxygen vacancies was taken and dispersed in 10 mL of an aqueous solution. A resulting dispersion was transferred to a photochemical reactor and irradiated under a 500 W mercury lamp for 2 h to obtain a black bismuth oxyhalide nanomaterial-dispersed system f.

(9-2-5) The black bismuth oxyhalide nanomaterial-dispersed system f was centrifuged at 10,000 r/min. A resulting precipitate was washed 4 times with water and ethanol to obtain a solid. The solid was dispersed in 10 mL of ethanol to obtain a dispersion.

(9-2-6) 10 mL of a solution of PVP in acetone (with a concentration of 50 mg/mL) was added to the dispersion obtained in step (5). A resulting mixed solution was continuously stirred for 24 h on a magnetic stirrer at 80 r/min. After a reaction was completed, a resulting mixture was centrifuged at 10,000 r/min, and a resulting precipitate was washed 4 times with water and ethanol to obtain a 2D bismuth oxyhalide nanomaterial with a large number of oxygen vacancies, which was stored at 4° C.

The TEM, DPBF degradation rate, photothermal conversion efficiency, cytotoxicity, CT value measurement, and in vivo CT imaging test results of the 2D bismuth oxyhalide composites with different numbers of oxygen vacancies obtained in Example 9 are basically the same as those of Example 1. XPS shows that a proportion of oxygen vacancies in the grey bismuth oxyhalide nanomaterial is 20% to 40% and a proportion of oxygen vacancies in the black bismuth oxyhalide nanomaterial is greater than 40%.

### Example 10

(10-2-1) 0.97 g of Bi(NO₃)₃.5H₂O and 0.315 g of BiCl₃ were weighed and dissolved in 40 mL of DEG, and a resulting mixed solution was continuously stirred for 10 min on a magnetic stirrer at 80 r/min and then subjected to an ultrasonic treatment for 10 min in an ultrasonic machine to obtain a solution d.

(10-2-2) The solution a was transferred to a PTFE-lined hydrothermal reactor to undergo a reaction at 180° C. for 6 h.

(10-2-3) After the reaction was completed, a resulting reaction solution was cooled to room temperature, and a resulting supernatant was removed. A resulting precipitate was washed 4 times with water and ethanol and then dried to obtain a grey bismuth oxyhalide material e with a small number of oxygen vacancies for later use.

(10-2-4) 50 mg of the grey bismuth oxyhalide material e with a small number of oxygen vacancies was taken and dispersed in 10 mL of an aqueous solution. A resulting dispersion was transferred to a photochemical reactor and irradiated under a 500 W mercury lamp for 2 h to obtain a black bismuth oxyhalide nanomaterial-dispersed system f.

(10-2-5) The black bismuth oxyhalide nanomaterial-dispersed system f was centrifuged at 10,000 r/min, a resulting precipitate was washed 4 times with water and ethanol to obtain a solid, and the solid was dispersed in 10 mL of ethanol to obtain a dispersion.

(10-2-6) 50 mL of a solution of PLGA in water was added to the dispersion obtained in step (5), and a resulting mixed solution was continuously stirred for 24 h on a magnetic stirrer at 80 r/min. After a reaction was completed, a resulting mixture was centrifuged at 10,000 r/min, and a resulting precipitate was washed 4 times with water and ethanol to obtain a 2D bismuth oxyhalide nanomaterial with a large number of oxygen vacancies, which was stored at 4° C.

The TEM, DPBF degradation rate, photothermal conversion efficiency, cytotoxicity, CT value measurement, and in vivo CT imaging test results of the 2D bismuth oxyhalide composites with different numbers of oxygen vacancies obtained in Example 10 are basically the same as those of Example 1. XPS shows that a proportion of oxygen vacancies in the grey bismuth oxyhalide nanomaterial is 20% to 40% and a proportion of oxygen vacancies in the black bismuth oxyhalide nanomaterial is greater than 40%.

### Example 11

(11-2-1) 0.97 g of Bi(NO₃)₃.5H₂O and 0.315 g of BiCl₃ were weighed and dissolved in 40 mL of DEG. A resulting mixed solution was continuously stirred for 10 min on a magnetic stirrer at 80 r/min and then subjected to an ultrasonic treatment for 10 min in an ultrasonic machine to obtain a solution d.

(11-2-2) The solution a was transferred to a PTFE-lined hydrothermal reactor to undergo a reaction at 180° C. for 6 h.

(11-2-3) After the reaction was completed, a resulting reaction solution was cooled to room temperature, and a resulting supernatant was removed. A resulting precipitate was washed 4 times with water and ethanol and then dried to obtain a grey bismuth oxyhalide material e with a small number of oxygen vacancies for later use.

(11-2-4) 50 mg of the grey bismuth oxyhalide material e with a small number of oxygen vacancies was taken and dispersed in 10 mL of an aqueous solution. A resulting dispersion was transferred to a photochemical reactor and irradiated under a 500 W mercury lamp for 2 h to obtain a black bismuth oxyhalide nanomaterial-dispersed system f.

(11-2-5) The black bismuth oxyhalide nanomaterial-dispersed system f was centrifuged at 10,000 r/min. A resulting precipitate was washed 4 times with water and ethanol to obtain a solid, and the solid was dispersed in 10 mL of ethanol to obtain a dispersion.

(11-2-6) 10 mL of a solution of arginine in EG (with a concentration of 50 mg/mL) was added to the dispersion obtained in step (5). A resulting mixed solution was continuously stirred for 24 h on a magnetic stirrer at 80 r/min. After a reaction was completed, a resulting mixture was centrifuged at 10,000 r/min, and a resulting precipitate was washed 4 times with water and ethanol to obtain a 2D bismuth oxyhalide nanomaterial with a large number of oxygen vacancies, which was stored at 4° C.

The TEM, DPBF degradation rate, photothermal conversion efficiency, cytotoxicity, CT value measurement, and in vivo CT imaging test results of the 2D bismuth oxyhalide composites with different numbers of oxygen vacancies obtained in Example 11 are basically the same as those of Example 1. XPS shows that a proportion of oxygen vacancies in the grey bismuth oxyhalide nanomaterial is 20% to 40% and a proportion of oxygen vacancies in the black bismuth oxyhalide nanomaterial is greater than 40%.

### Example 12

(12-2-1) 0.97 g of Bi(NO₃)₃.5H₂O and 0.315 g of BiF₃ were weighed and dissolved in 40 mL of DEG. A resulting mixed solution was continuously stirred for 10 min on a magnetic stirrer at 80 r/min and then subjected to an ultrasonic treatment for 10 min in an ultrasonic machine to obtain a solution d.

(12-2-2) The solution a was transferred to a PTFE-lined hydrothermal reactor to undergo a reaction at 180° C. for 6 h.

(12-2-3) After the reaction was completed, a resulting reaction solution was cooled to room temperature, and a resulting supernatant was removed. A resulting precipitate was washed 4 times with water and ethanol and then dried to obtain a grey bismuth oxyhalide material e with a small number of oxygen vacancies for later use.

(12-2-4) 50 mg of the grey bismuth oxyhalide material e with a small number of oxygen vacancies was taken and dispersed in 10 mL of an aqueous solution. A resulting dispersion was transferred to a photochemical reactor and irradiated under a 500 W mercury lamp for 2 h to obtain a black bismuth oxyhalide nanomaterial-dispersed system f.

(12-2-5) The black bismuth oxyhalide nanomaterial-dispersed system f was centrifuged at 10,000 r/min. A resulting precipitate was washed 4 times with water and ethanol to obtain a solid, and the solid was dispersed in 10 mL of ethanol to obtain a dispersion.

(12-2-6) 10 mL of a solution of arginine in EG (with a concentration of 50 mg/mL) was added to the dispersion obtained in step (5). A resulting mixed solution was continuously stirred for 24 h on a magnetic stirrer at 80 r/min. After a reaction was completed, a resulting mixture was centrifuged at 10,000 r/min. A resulting precipitate was washed 4 times with water and ethanol to obtain a 2D bismuth oxyhalide nanomaterial with a large number of oxygen vacancies, which was stored at 4° C.

Results

The TEM, DPBF degradation rate, photothermal conversion efficiency, cytotoxicity, CT value measurement, and in vivo CT imaging test results of the 2D bismuth oxyhalide composites with different numbers of oxygen vacancies obtained in Example 12 are basically the same as those of Example 1. XPS shows that a proportion of oxygen vacancies in the grey bismuth oxyhalide nanomaterial is 20% to 40% and a proportion of oxygen vacancies in the black bismuth oxyhalide nanomaterial is greater than 40%.

### Example 13

(13-2-1) 0.97 g of Bi(NO₃)₃.5H₂O and 0.315 g of BiAt₃ were weighed and dissolved in 40 mL of DEG. A resulting mixed solution was continuously stirred for 10 min on a magnetic stirrer at 80 r/min and then subjected to an ultrasonic treatment for 10 min in an ultrasonic machine to obtain a solution d.

(13-2-2) The solution a was transferred to a PTFE-lined hydrothermal reactor to undergo a reaction at 180° C. for 6 h.

(13-2-3) After the reaction was completed, a resulting reaction solution was cooled to room temperature. A resulting supernatant was removed, and a resulting precipitate was washed 4 times with water and ethanol and then dried to obtain a grey bismuth oxyhalide material e with a small number of oxygen vacancies for later use.

(13-2-4) 50 mg of the grey bismuth oxyhalide material e with a small number of oxygen vacancies was taken and dispersed in 10 mL of an aqueous solution. A resulting dispersion was transferred to a photochemical reactor and irradiated under a 500 W mercury lamp for 2 h to obtain a black bismuth oxyhalide nanomaterial-dispersed system f.

(13-2-5) The black bismuth oxyhalide nanomaterial-dispersed system f was centrifuged at 10,000 r/min. A resulting precipitate was washed 4 times with water and ethanol to obtain a solid, and the solid was dispersed in 10 mL of ethanol to obtain a dispersion.

(13-2-6) 10 mL of a solution of arginine in EG (with a concentration of 50 mg/mL) was added to the dispersion obtained in step (5), and a resulting mixed solution was continuously stirred for 24 h on a magnetic stirrer at 80 r/min. After a reaction was completed, a resulting mixture was centrifuged at 10,000 r/min. A resulting precipitate was washed 4 times with water and ethanol to obtain a 2D bismuth oxyhalide nanomaterial with a large number of oxygen vacancies, which was stored at 4° C.

Results

The TEM, DPBF degradation rate, photothermal conversion efficiency, cytotoxicity, CT value measurement, and in vivo CT imaging test results of the 2D bismuth oxyhalide composites with different numbers of oxygen vacancies obtained in Example 13 are basically the same as those of Example 1. XPS shows that a proportion of oxygen vacancies in the grey bismuth oxyhalide nanomaterial is 20% to 40% and a proportion of oxygen vacancies in the black bismuth oxyhalide nanomaterial is greater than 40%.

### Example 14

Preparation of a Grey Bismuth Oxyhalide Material with a Small Number of Oxygen Vacancies and a Black Bismuth Oxyhalide Nanomaterial with a Large Number of Oxygen Vacancies

(14-2-1) 0.71 g of Bi₂(SO₄)₃ and 0.315 g of BiCl₃ were weighed and dissolved in 40 mL of DEG. A resulting mixed solution was continuously stirred for 10 min on a magnetic stirrer at 80 r/min and then subjected to an ultrasonic treatment for 10 min using an ultrasonic machine to obtain a solution d.

(14-2-2) The solution d was transferred to a PTFE-lined hydrothermal reactor to undergo a reaction at 160° C. for 12 h.

(14-2-3) After the reaction was completed, a resulting reaction solution was cooled to room temperature. A resulting supernatant was removed, and a resulting precipitate was washed 4 times with water and ethanol, and then dried to obtain a grey bismuth oxyhalide material e with a small number of oxygen vacancies for later use, which was denoted as sample 14-2.

(14-2-4) 50 mg of the grey bismuth oxyhalide material e with a small number of oxygen vacancies was taken and dispersed in 10 mL of an aqueous solution. A resulting dispersion was transferred to a photochemical reactor and irradiated under a 500 W mercury lamp for 2 h to obtain a black bismuth oxyhalide nanomaterial-dispersed system f.

(14-2-5) The black bismuth oxyhalide nanomaterial-dispersed system f was centrifuged at 10,000 r/min. A resulting precipitate was washed 4 times with water and ethanol to obtain a solid, which was denoted as sample 14-3. The solid was dispersed in 10 mL of ethanol to obtain a dispersion.

(14-2-6) 10 mL of a solution of PEG in ethanol (with a concentration of 50 mg/mL) was added to the dispersion obtained in step (5). A resulting mixed solution was continuously stirred for 24 h on a magnetic stirrer at 80 r/min. After a reaction was completed, a resulting mixture was centrifuged at 10,000 r/min. A resulting precipitate was washed 4 times with water and ethanol to obtain a black 2D bismuth oxyhalide nanomaterial with a large number of oxygen vacancies, which was denoted as sample 14-4 and stored at 4° C.

Results

The TEM, DPBF degradation rate, photothermal conversion efficiency, cytotoxicity, CT value measurement, and in vivo CT imaging test results of the 2D bismuth oxyhalide composites with different numbers of oxygen vacancies obtained in Example 14 are basically the same as those of Example 1. XPS shows that a proportion of oxygen vacancies in the grey bismuth oxyhalide nanomaterial is 20% to 40% and a proportion of oxygen vacancies in the black bismuth oxyhalide nanomaterial is greater than 40%.

### Example 15

Preparation of a Grey Bismuth Oxyhalide Material with a Small Number of Oxygen Vacancies and a Black Bismuth Oxyhalide Nanomaterial with a Large Number of Oxygen Vacancies

(15-2-1) 0.47 g of Bi₂O₃ and 0.315 g of BiCl₃ were weighed and dissolved in 40 mL of DEG. A resulting mixed solution was continuously stirred for 10 min on a magnetic stirrer at 80 r/min and then subjected to an ultrasonic treatment for 10 min in an ultrasonic machine to obtain a solution d.

(15-2-2) The solution d was transferred to a PTFE-lined hydrothermal reactor to undergo a reaction at 160° C. for 12 h.

(15-2-3) After the reaction was completed, a resulting reaction solution was cooled to room temperature, and a resulting supernatant was removed. A resulting precipitate was washed 4 times with water and ethanol and then dried to obtain a grey bismuth oxyhalide material e with a small number of oxygen vacancies for later use, which was denoted as sample 15-2.

(15-2-4) 50 mg of the grey bismuth oxyhalide material e with a small number of oxygen vacancies was taken and dispersed in 10 mL of an aqueous solution. A resulting dispersion was transferred to a photochemical reactor and irradiated under a 500 W mercury lamp for 2 h to obtain a black bismuth oxyhalide nanomaterial-dispersed system f.

(15-2-5) The black bismuth oxyhalide nanomaterial-dispersed system f was centrifuged at 10,000 r/min. A resulting precipitate was washed 4 times with water and ethanol to obtain a solid, which was denoted as sample 15-3. The solid was dispersed in 10 mL of ethanol to obtain a dispersion.

(15-2-6) 10 mL of a solution of PEG in ethanol (with a concentration of 50 mg/mL) was added to the dispersion obtained in step (5). A resulting mixed solution was continuously stirred for 24 h on a magnetic stirrer at 80 r/min. After a reaction was completed, a resulting mixture was centrifuged at 10,000 r/min, and a resulting precipitate was washed 4 times with water and ethanol to obtain a black 2D bismuth oxyhalide nanomaterial with a large number of oxygen vacancies, which was denoted as sample 15-4, and stored at 4° C.

Results

The TEM, DPBF degradation rate, photothermal conversion efficiency, cytotoxicity, CT value measurement, and in vivo CT imaging test results of the 2D bismuth oxyhalide composites with different numbers of oxygen vacancies obtained in Example 15 are basically the same as those of Example 1. XPS shows that a proportion of oxygen vacancies in the grey bismuth oxyhalide nanomaterial is 20% to 40% and a proportion of oxygen vacancies in the black bismuth oxyhalide nanomaterial is greater than 40%.

### Example 16

Preparation of a Grey Bismuth Oxyhalide Material with a Small Number of Oxygen Vacancies and a Black Bismuth Oxyhalide Nanomaterial with a Large Number of Oxygen Vacancies

(16-2-1) 0.61 g of BiPO₄ and 0.315 g of BiCl₃ were weighed and dissolved in 40 mL of DEG. A resulting mixed solution was continuously stirred for 10 min on a magnetic stirrer at 80 r/min and then subjected to an ultrasonic treatment for 10 min in an ultrasonic machine to obtain a solution d.

(16-2-2) The solution d was transferred to a PTFE-lined hydrothermal reactor to undergo a reaction at 160° C. for 12 h.

(16-2-3) After the reaction was completed, a resulting reaction solution was cooled to room temperature, and a resulting supernatant was removed. A resulting precipitate was washed 4 times with water and ethanol and then dried to obtain a grey bismuth oxyhalide material e with a small number of oxygen vacancies for later use, which was denoted as sample 16-2.

(16-2-4) 50 mg of the grey bismuth oxyhalide material e with a small number of oxygen vacancies was taken and dispersed in 10 mL of an aqueous solution. A resulting dispersion was transferred to a photochemical reactor and irradiated under a 500 W mercury lamp for 2 h to obtain a black bismuth oxyhalide nanomaterial-dispersed system f.

(16-2-5) The black bismuth oxyhalide nanomaterial-dispersed system f was centrifuged at 10,000 r/min, and a resulting precipitate was washed 4 times with water and ethanol to obtain a solid, which was denoted as sample 16-3. The solid was dispersed in 10 mL of ethanol to obtain a dispersion.

(16-2-6) 10 mL of a solution of PEG in ethanol (with a concentration of 50 mg/mL) was added to the dispersion obtained in step (5). A resulting mixed solution was continuously stirred for 24 h on a magnetic stirrer at 80 r/min. After a reaction was completed, a resulting mixture was centrifuged at 10,000 r/min. A resulting precipitate was washed 4 times with water and ethanol to obtain a black 2D bismuth oxyhalide nanomaterial with a large number of oxygen vacancies, which was denoted as sample 16-4 and stored at 4° C.

Results

The TEM, DPBF degradation rate, photothermal conversion efficiency, cytotoxicity, CT value measurement, and in vivo CT imaging test results of the 2D bismuth oxyhalide composites with different numbers of oxygen vacancies obtained in Example 16 are basically the same as those of Example 1. XPS shows that a proportion of oxygen vacancies in the grey bismuth oxyhalide nanomaterial is 20% to 40% and a proportion of oxygen vacancies in the black bismuth oxyhalide nanomaterial is greater than 40%.

### Example 17

Preparation of a Grey Bismuth Oxyhalide Material with a Small Number of Oxygen Vacancies and a Black Bismuth Oxyhalide Nanomaterial with a Large Number of Oxygen Vacancies

(17-2-1) 1.41 g of (BiO)₂CO₃.½H₂O and 0.315 g of BiCl₃ were weighed and dissolved in 40 mL of DEG. A resulting mixed solution was continuously stirred for 10 min on a magnetic stirrer at 80 r/min and then subjected to an ultrasonic treatment for 10 min in an ultrasonic machine to obtain a solution d.

(17-2-2) The solution d was transferred to a PTFE-lined hydrothermal reactor to undergo a reaction at 160° C. for 12 h.

(17-2-3) After the reaction was completed, a resulting reaction solution was cooled to room temperature, and a resulting supernatant was removed. A resulting precipitate was washed 4 times with water and ethanol and then dried to obtain a grey bismuth oxyhalide material e with a small number of oxygen vacancies for later use, which was denoted as sample 17-2.

(17-2-4) 50 mg of the grey bismuth oxyhalide material e with a small number of oxygen vacancies was taken and dispersed in 10 mL of an aqueous solution. A resulting dispersion was transferred to a photochemical reactor and irradiated under a 500 W mercury lamp for 2 h to obtain a black bismuth oxyhalide nanomaterial-dispersed system f.

(17-2-5) The black bismuth oxyhalide nanomaterial-dispersed system f was centrifuged at 10,000 r/min. A resulting precipitate was washed 4 times with water and ethanol to obtain a solid, which was denoted as sample 17-3. The solid was dispersed in 10 mL of ethanol to obtain a dispersion.

(17-2-6) 10 mL of a solution of PEG in ethanol (with a concentration of 50 mg/mL) was added to the dispersion obtained in step (5). A resulting mixed solution was continuously stirred for 24 h on a magnetic stirrer at 80 r/min. After a reaction was completed, a resulting mixture was centrifuged at 10,000 r/min, and a resulting precipitate was washed 4 times with water and ethanol to obtain a black 2D bismuth oxyhalide nanomaterial with a large number of oxygen vacancies, which was denoted as sample 17-4 and stored at 4° C.

Results

The TEM, DPBF degradation rate, photothermal conversion efficiency, cytotoxicity, CT value measurement, and in vivo CT imaging test results of the 2D bismuth oxyhalide composites with different numbers of oxygen vacancies obtained in Example 17 are basically the same as those of Example 1. XPS shows that a proportion of oxygen vacancies in the grey bismuth oxyhalide nanomaterial is 20% to 40% and a proportion of oxygen vacancies in the black bismuth oxyhalide nanomaterial is greater than 40%.

### Example 18

Preparation of a Grey Bismuth Oxyhalide Material with a Small Number of Oxygen Vacancies and a Black Bismuth Oxyhalide Nanomaterial with a Large Number of Oxygen Vacancies

(18-2-1) 0.62 g of BiFeO₃.½H₂O and 0.315 g of BiCl₃ were weighed and dissolved in 40 mL of DEG. A resulting mixed solution was continuously stirred for 10 min on a magnetic stirrer at 80 r/min and then subjected to an ultrasonic treatment for 10 min in an ultrasonic machine to obtain a solution d.

(18-2-2) The solution d was transferred to a PTFE-lined hydrothermal reactor to undergo a reaction at 160° C. for 12 h.

(18-2-3) After the reaction was completed, a resulting reaction solution was cooled to room temperature, and a resulting supernatant was removed. A resulting precipitate was washed 4 times with water and ethanol and then dried to obtain a grey bismuth oxyhalide material e with a small number of oxygen vacancies for later use, which was denoted as sample 18-2.

(18-2-4) 50 mg of the grey bismuth oxyhalide material e with a small number of oxygen vacancies was taken and dispersed in 10 mL of an aqueous solution. A resulting dispersion was transferred to a photochemical reactor and irradiated under a 500 W mercury lamp for 2 h to obtain a black bismuth oxyhalide nanomaterial-dispersed system f.

(18-2-5) The black bismuth oxyhalide nanomaterial-dispersed system f was centrifuged at 10,000 r/min. A resulting precipitate was washed 4 times with water and ethanol to obtain a solid, which was denoted as sample 18-3. The solid was dispersed in 10 mL of ethanol to obtain a dispersion.

(18-2-6) 10 mL of a solution of PEG in ethanol (with a concentration of 50 mg/mL) was added to the dispersion obtained in step (5), and a resulting mixed solution was continuously stirred for 24 h on a magnetic stirrer at 80 r/min. After a reaction was completed, a resulting mixture was centrifuged at 10,000 r/min, and a resulting precipitate was washed 4 times with water and ethanol to obtain a black 2D bismuth oxyhalide nanomaterial with a large number of oxygen vacancies, which was denoted as sample 18-4, and stored at 4° C.

Results

The TEM, DPBF degradation rate, photothermal conversion efficiency, cytotoxicity, CT value measurement, and in vivo CT imaging test results of the 2D bismuth oxyhalide composites with different numbers of oxygen vacancies obtained in Example 18 are basically the same as those of Example 1. XPS shows that a proportion of oxygen vacancies in the grey bismuth oxyhalide nanomaterial is 20% to 40% and a proportion of oxygen vacancies in the black bismuth oxyhalide nanomaterial is greater than 40%.

### Example 19

Preparation of a Grey Bismuth Oxyhalide Material with a Small Number of Oxygen Vacancies and a Black Bismuth Oxyhalide Nanomaterial with a Large Number of Oxygen Vacancies

(19-2-1) 0.97 g of Bi(NO₃)₃.5H₂O and 0.266 g of BiF₃ were weighed and dissolved in 40 mL of DEG, and a resulting mixed solution was continuously stirred for 10 min on a magnetic stirrer at 80 r/min and then subjected to an ultrasonic treatment for 10 min in an ultrasonic machine to obtain a solution d.

(19-2-2) The solution d was transferred to a PTFE-lined hydrothermal reactor to undergo a reaction at 160° C. for 12 h.

(19-2-3) After the reaction was completed, a resulting reaction solution was cooled to room temperature, and a resulting supernatant was removed. A resulting precipitate was washed 4 times with water and ethanol and then dried to obtain a grey bismuth oxyhalide material e with a small number of oxygen vacancies for later use, which was denoted as sample 19-2.

(19-2-4) 50 mg of the grey bismuth oxyhalide material e with a small number of oxygen vacancies was taken and dispersed in 10 mL of an aqueous solution. A resulting dispersion was transferred to a photochemical reactor and irradiated under a 500 W mercury lamp for 2 h to obtain a black bismuth oxyhalide nanomaterial-dispersed system f.

(19-2-5) The black bismuth oxyhalide nanomaterial-dispersed system f was centrifuged at 10,000 r/min. A resulting precipitate was washed 4 times with water and ethanol to obtain a solid, which was denoted as sample 19-3. The solid was dispersed in 10 mL of ethanol to obtain a dispersion.

(19-2-6) 10 mL of a solution of PEG in ethanol (with a concentration of 50 mg/mL) was added to the dispersion obtained in step (5). A resulting mixed solution was continuously stirred for 24 h on a magnetic stirrer at 80 r/min. After a reaction was completed, a resulting mixture was centrifuged at 10,000 r/min. A resulting precipitate was washed 4 times with water and ethanol to obtain a black 2D bismuth oxyhalide nanomaterial with a large number of oxygen vacancies, which was denoted as sample 19-4, and stored at 4° C.

Results

The TEM, DPBF degradation rate, photothermal conversion efficiency, cytotoxicity, CT value measurement, and in vivo CT imaging test results of the 2D bismuth oxyhalide composites with different numbers of oxygen vacancies obtained in Example 19 are basically the same as those of Example 1. XPS shows that a proportion of oxygen vacancies in the grey bismuth oxyhalide nanomaterial is 20% to 40% and a proportion of oxygen vacancies in the black bismuth oxyhalide nanomaterial is greater than 40%.

### Example 20

Preparation of a Grey Bismuth Oxyhalide Material with a Small Number of Oxygen Vacancies and a Black Bismuth Oxyhalide Nanomaterial with a Large Number of Oxygen Vacancies

(20-2-1) 0.97 g of Bi(NO₃)₃.5H₂O and 0.449 g of BiBr₃ were weighed and dissolved in 40 mL of DEG. A resulting mixed solution was continuously stirred for 10 min on a magnetic stirrer at 80 r/min and then subjected to an ultrasonic treatment for 10 min in an ultrasonic machine to obtain a solution d.

(20-2-2) The solution d was transferred to a PTFE-lined hydrothermal reactor to undergo a reaction at 160° C. for 12 h.

(20-2-3) After the reaction was completed, a resulting reaction solution was cooled to room temperature, and a resulting supernatant was removed. A resulting precipitate was washed 4 times with water and ethanol and then dried to obtain a grey bismuth oxyhalide material e with a small number of oxygen vacancies for later use, which was denoted as sample 20-2.

(20-2-4) 50 mg of the grey bismuth oxyhalide material e with a small number of oxygen vacancies was taken and dispersed in 10 mL of an aqueous solution. A resulting dispersion was transferred to a photochemical reactor and irradiated under a 500 W mercury lamp for 2 h to obtain a black bismuth oxyhalide nanomaterial-dispersed system f.

(20-2-5) The black bismuth oxyhalide nanomaterial-dispersed system f was centrifuged at 10,000 r/min. A resulting precipitate was washed 4 times with water and ethanol to obtain a solid, which was denoted as sample 20-3. The solid was dispersed in 10 mL of ethanol to obtain a dispersion.

(20-2-6) 10 mL of a solution of PEG in ethanol (with a concentration of 50 mg/mL) was added to the dispersion obtained in step (5). A resulting mixed solution was continuously stirred for 24 h on a magnetic stirrer at 80 r/min. After a reaction was completed, a resulting mixture was centrifuged at 10,000 r/min. A resulting precipitate was washed 4 times with water and ethanol to obtain a black 2D bismuth oxyhalide nanomaterial with a large number of oxygen vacancies, which was denoted as sample 20-4, and stored at 4° C.

Results

The TEM, DPBF degradation rate, photothermal conversion efficiency, cytotoxicity, CT value measurement, and in vivo CT imaging test results of the 2D bismuth oxyhalide composites with different numbers of oxygen vacancies obtained in Example 20 are basically the same as those of Example 1. XPS shows that a proportion of oxygen vacancies in the grey bismuth oxyhalide nanomaterial is 20% to 40% and a proportion of oxygen vacancies in the black bismuth oxyhalide nanomaterial is greater than 40%.

### Example 21

Preparation of a Grey Bismuth Oxyhalide Material with a Small Number of Oxygen Vacancies and a Black Bismuth Oxyhalide Nanomaterial with a Large Number of Oxygen Vacancies

(21-2-1) 0.97 g of Bi(NO₃)₃.5H₂O and 0.59 g of BiI₃ were weighed and dissolved in 40 mL of DEG, and a resulting mixed solution was continuously stirred for 10 min on a magnetic stirrer at 80 r/min and then subjected to an ultrasonic treatment for 10 min in an ultrasonic machine to obtain a solution d.

(21-2-2) The solution d was transferred to a PTFE-lined hydrothermal reactor to undergo a reaction at 160° C. for 12 h.

(21-2-3) After the reaction was completed, a resulting reaction solution was cooled to room temperature, a resulting supernatant was removed, and a resulting precipitate was washed 4 times with water and ethanol and then dried to obtain a grey bismuth oxyhalide material e with a small number of oxygen vacancies for later use, which was denoted as sample 21-2.

(21-2-4) 50 mg of the grey bismuth oxyhalide material e with a small number of oxygen vacancies was taken and dispersed in 10 mL of an aqueous solution, and a resulting dispersion was transferred to a photochemical reactor and irradiated under a 500 W mercury lamp for 2 h to obtain a black bismuth oxyhalide nanomaterial-dispersed system f.

(21-2-5) The black bismuth oxyhalide nanomaterial-dispersed system f was centrifuged at 10,000 r/min, and a resulting precipitate was washed 4 times with water and ethanol to obtain a solid, which was denoted as sample 21-3; the solid was dispersed in 10 mL of ethanol to obtain a dispersion.

(21-2-6) 10 mL of a solution of PEG in ethanol (with a concentration of 50 mg/mL) was added to the dispersion obtained in step (5), and a resulting mixed solution was continuously stirred for 24 h on a magnetic stirrer at 80 r/min. After a reaction was completed, a resulting mixture was centrifuged at 10,000 r/min, and a resulting precipitate was washed 4 times with water and ethanol to obtain a black 2D bismuth oxyhalide nanomaterial with a large number of oxygen vacancies, which was denoted as sample 21-4, and stored at 4° C.

Results

The TEM, DPBF degradation rate, photothermal conversion efficiency, cytotoxicity, CT value measurement, and in vivo CT imaging test results of the 2D bismuth oxyhalide composites with different numbers of oxygen vacancies obtained in Example 21 are basically the same as those of Example 1. XPS shows that a proportion of oxygen vacancies in the grey bismuth oxyhalide nanomaterial is 20% to 40% and a proportion of oxygen vacancies in the black bismuth oxyhalide nanomaterial is greater than 40%.

### Example 22

Preparation of a Grey Bismuth Oxyhalide Material with a Small Number of Oxygen Vacancies and a Black Bismuth Oxyhalide Nanomaterial with a Large Number of Oxygen Vacancies

(22-2-1) 0.97 g of Bi(NO₃)₃.5H₂O and 0.83 g of BiAt₃ were weighed and dissolved in 40 mL of DEG, and a resulting mixed solution was continuously stirred for 10 min on a magnetic stirrer at 80 r/min and then subjected to an ultrasonic treatment for 10 min in an ultrasonic machine to obtain a solution d.

(22-2-2) The solution d was transferred to a PTFE-lined hydrothermal reactor to undergo a reaction at 160° C. for 12 h.

(22-2-3) After the reaction was completed, a resulting reaction solution was cooled to room temperature, a resulting supernatant was removed, and a resulting precipitate was washed 4 times with water and ethanol and then dried to obtain a grey bismuth oxyhalide material e with a small number of oxygen vacancies for later use, which was denoted as sample 21-2.

(22-2-4) 50 mg of the grey bismuth oxyhalide material e with a small number of oxygen vacancies was taken and dispersed in 10 mL of an aqueous solution, and a resulting dispersion was transferred to a photochemical reactor and irradiated under a 500 W mercury lamp for 2 h to obtain a black bismuth oxyhalide nanomaterial-dispersed system f.

(22-2-5) The black bismuth oxyhalide nanomaterial-dispersed system f was centrifuged at 10,000 r/min, and a resulting precipitate was washed 4 times with water and ethanol to obtain a solid, which was denoted as sample 22-3; the solid was dispersed in 10 mL of ethanol to obtain a dispersion.

(22-2-6) 10 mL of a solution of PEG in ethanol (with a concentration of 50 mg/mL) was added to the dispersion obtained in step (5), and a resulting mixed solution was continuously stirred for 24 h on a magnetic stirrer at 80 r/min. After a reaction was completed, a resulting mixture was centrifuged at 10,000 r/min, and a resulting precipitate was washed 4 times with water and ethanol to obtain a black 2D bismuth oxyhalide nanomaterial with a large number of oxygen vacancies, which was denoted as sample 22-4 and stored at 4° C.

Results

The TEM, DPBF degradation rate, photothermal conversion efficiency, cytotoxicity, CT value measurement, and in vivo CT imaging test results of the 2D bismuth oxyhalide composites with different numbers of oxygen vacancies obtained in Example 22 are basically the same as those of Example 1. XPS shows that a proportion of oxygen vacancies in the grey bismuth oxyhalide nanomaterial is 20% to 40% and a proportion of oxygen vacancies in the black bismuth oxyhalide nanomaterial is greater than 40%.

### Example 23

Preparation of a Grey Bismuth Oxyhalide Material with a Small Number of Oxygen Vacancies and a Black Bismuth Oxyhalide Nanomaterial with a Large Number of Oxygen Vacancies

(23-2-1) 0.97 g of Bi(NO₃)₃.5H₂O and 0.59 g of BiI₃ were weighed and dissolved in 40 mL of DEG, and a resulting mixed solution was continuously stirred for 10 min on a magnetic stirrer at 80 r/min and then subjected to an ultrasonic treatment for 10 min in an ultrasonic machine to obtain a solution d.

(23-2-2) The solution d was transferred to a PTFE-lined hydrothermal reactor to undergo a reaction at 120° C. for 6 h.

(23-2-3) After the reaction was completed, a resulting reaction solution was cooled to room temperature, a resulting supernatant was removed, and a resulting precipitate was washed 4 times with water and ethanol and then dried to obtain a grey bismuth oxyhalide material e with a small number of oxygen vacancies for later use, which was denoted as sample 7-2.

(23-2-4) 50 mg of the grey bismuth oxyhalide material e with a small number of oxygen vacancies was taken and mixed with 2 g of dithizone, and a resulting powdery mixture was transferred to a tube furnace and calcined at 400° C. for 2 h to obtain a bismuth oxyhalide nanomaterial d with a large number of oxygen vacancies.

(23-2-5) The bismuth oxyhalide nanomaterial d was washed 8 times with water and ethanol to obtain a solid, which was denoted as sample 23-3; the solid was dispersed in 10 mL of ethanol to obtain a dispersion.

(23-2-6) 10 mL of a solution of PLGA in ethanol (with a concentration of 25 mg/mL) was added to the dispersion obtained in step (5), and a resulting mixed solution was continuously stirred for 24 h on a magnetic stirrer at 80 r/min. After a reaction was completed, a resulting mixture was centrifuged at 10,000 r/min, and a resulting precipitate was washed 4 times with water and ethanol to obtain a black 2D bismuth oxyhalide nanomaterial with a large number of oxygen vacancies, which was denoted as sample 23-4, and stored at 4° C.

Results

The TEM, DPBF degradation rate, photothermal conversion efficiency, cytotoxicity, CT value measurement, and in vivo CT imaging test results of the 2D bismuth oxyhalide composites with different numbers of oxygen vacancies obtained in Example 23 are basically the same as those of Example 1. XPS shows that a proportion of oxygen vacancies in the grey bismuth oxyhalide nanomaterial is 20% to 40% and a proportion of oxygen vacancies in the black bismuth oxyhalide nanomaterial is greater than 40%.

### Example 24

Preparation of a Grey Bismuth Oxyhalide Material with a Small Number of Oxygen Vacancies and a Black Bismuth Oxyhalide Nanomaterial with a Large Number of Oxygen Vacancies

(24-2-1) 0.97 g of Bi(NO₃)₃.5H₂O and 0.315 g of BiCl₃ were weighed and dissolved in 40 mL of DEG, and a resulting mixed solution was continuously stirred for 20 min on a magnetic stirrer at 80 r/min and then subjected to an ultrasonic treatment for 20 min in an ultrasonic machine to obtain a solution d.

(24-2-2) The solution d was transferred to a PTFE-lined hydrothermal reactor to undergo a reaction at 180° C. for 6 h.

(24-2-3) After the reaction was completed, a resulting reaction solution was cooled to room temperature, a resulting supernatant was removed, and a resulting precipitate was washed 4 times with water and ethanol and then dried to obtain a grey bismuth oxyhalide material e with a small number of oxygen vacancies for later use, which was denoted as sample 24-2.

(24-2-4) 50 mg of the grey bismuth oxyhalide material e with a small number of oxygen vacancies was taken and mixed with 2 g of dithizone, and a resulting powdery mixture was transferred to a tube furnace and calcined at 400° C. for 4 h to obtain a bismuth oxyhalide nanomaterial d with a large number of oxygen vacancies.

(24-2-5) The bismuth oxyhalide nanomaterial d was washed 8 times with water and ethanol to obtain a solid, which was denoted as sample 24-3; the solid was dispersed in 10 mL of ethanol to obtain a dispersion.

(24-2-6) 20 mL of a solution of arginine in ethanol (with a concentration of 25 mg/mL) was added to the dispersion obtained in step (5), and a resulting mixed solution was continuously stirred for 24 h on a magnetic stirrer at 160 r/min. After a reaction was completed, a resulting mixture was centrifuged at 10,000 r/min, and a resulting precipitate was washed 4 times with water and ethanol to obtain a black 2D bismuth oxyhalide nanomaterial with a large number of oxygen vacancies, which was denoted as sample 24-4, and stored at 4° C.

Results

The TEM, DPBF degradation rate, photothermal conversion efficiency, cytotoxicity, CT value measurement, and in vivo CT imaging test results of the 2D bismuth oxyhalide composites with different numbers of oxygen vacancies obtained in Example 24 are basically the same as those of Example 1. XPS shows that a proportion of oxygen vacancies in the grey bismuth oxyhalide nanomaterial is 20% to 40% and a proportion of oxygen vacancies in the black bismuth oxyhalide nanomaterial is greater than 40%.

### Example 25

Preparation of a Grey Bismuth Oxyhalide Material with a Small Number of Oxygen Vacancies and a Black Bismuth Oxyhalide Nanomaterial with a Large Number of Oxygen Vacancies

(25-2-1) 0.97 g of Bi(NO₃)₃.5H₂O and 0.315 g of BiCl₃ were weighed and dissolved in 40 mL of ethanol, and a resulting mixed solution was continuously stirred for 10 min on a magnetic stirrer at 80 r/min and then subjected to an ultrasonic treatment for 10 min in an ultrasonic machine to obtain a solution d.

(25-2-2) The solution d was transferred to a PTFE-lined hydrothermal reactor to undergo a reaction at 140° C. for 26 h.

(25-2-3) After the reaction was completed, a resulting reaction solution was cooled to room temperature, a resulting supernatant was removed, and a resulting precipitate was washed 4 times with water and ethanol and then dried to obtain a grey bismuth oxyhalide material e with a small number of oxygen vacancies for later use, which was denoted as sample 25-2.

(25-2-4) 50 mg of the grey bismuth oxyhalide material e with a small number of oxygen vacancies was taken and mixed with 2 g of dithizone, and a resulting powdery mixture was transferred to a tube furnace and calcined at 400° C. for 8 h to obtain a bismuth oxyhalide nanomaterial d with a large number of oxygen vacancies.

(25-2-5) The bismuth oxyhalide nanomaterial d was washed 8 times with water and ethanol to obtain a solid, which was denoted as sample 25-3; the solid was dispersed in 10 mL of ethanol to obtain a dispersion.

(25-2-6) 10 mL of a solution of PVP in DEG (with a concentration of 25 mg/mL) was added to the dispersion obtained in step (5), and a resulting mixed solution was continuously stirred for 24 h on a magnetic stirrer at 80 r/min. After a reaction was completed, a resulting mixture was centrifuged at 10,000 r/min, and a resulting precipitate was washed 4 times with water and ethanol to obtain a black 2D bismuth oxyhalide nanomaterial with a large number of oxygen vacancies, which was denoted as sample 25-4, and stored at 4° C.

Results

The TEM, DPBF degradation rate, photothermal conversion efficiency, cytotoxicity, CT value measurement, and in vivo CT imaging test results of the 2D bismuth oxyhalide composites with different numbers of oxygen vacancies obtained in Example 25 are basically the same as those of Example 1. XPS shows that a proportion of oxygen vacancies in the grey bismuth oxyhalide nanomaterial is 20% to 40% and a proportion of oxygen vacancies in the black bismuth oxyhalide nanomaterial is greater than 40%.

### Example 26

Preparation of a Grey Bismuth Oxyhalide Material with a Small Number of Oxygen Vacancies and a Black Bismuth Oxyhalide Nanomaterial with a Large Number of Oxygen Vacancies

(26-2-1) 0.97 g of Bi(NO₃)₃.5H₂O and 0.315 g of BiCl₃ were weighed and dissolved in 40 mL of DEG, and a resulting mixed solution was continuously stirred for 10 min on a magnetic stirrer at 80 r/min and then subjected to an ultrasonic treatment for 10 min in an ultrasonic machine to obtain a solution d.

(26-2-2) The solution d was transferred to a PTFE-lined hydrothermal reactor to undergo a reaction at 140° C. for 6 h.

(26-2-3) After the reaction was completed, a resulting reaction solution was cooled to room temperature, a resulting supernatant was removed, and a resulting precipitate was washed 4 times with water and ethanol and then dried to obtain a grey bismuth oxyhalide material e with a small number of oxygen vacancies for later use, which was denoted as sample 26-2.

(26-2-4) 50 mg of the grey bismuth oxyhalide material e with a small number of oxygen vacancies was taken and mixed with 2 g of SBH, and a resulting powdery mixture was transferred to a tube furnace and calcined at 400° C. for 2 h to obtain a bismuth oxyhalide nanomaterial d with a large number of oxygen vacancies.

(26-2-5) The bismuth oxyhalide nanomaterial d was washed 8 times with water and ethanol to obtain a solid, which was denoted as sample 26-3; the solid was dispersed in 10 mL of ethanol to obtain a dispersion.

(26-2-6) 10 mL of a solution of PVP in ethanol (with a concentration of 25 mg/mL) was added to the dispersion obtained in step (5), and a resulting mixed solution was continuously stirred for 24 h on a magnetic stirrer at 80 r/min. After a reaction was completed, a resulting mixture was centrifuged at 10,000 r/min, and a resulting precipitate was washed 4 times with water and ethanol to obtain a black 2D bismuth oxyhalide nanomaterial with a large number of oxygen vacancies, which was denoted as sample 26-4, and stored at 4° C.

Results

The TEM, DPBF degradation rate, photothermal conversion efficiency, cytotoxicity, CT value measurement, and in vivo CT imaging test results of the 2D bismuth oxyhalide composites with different numbers of oxygen vacancies obtained in Example 26 are basically the same as those of Example 1. XPS shows that a proportion of oxygen vacancies in the grey bismuth oxyhalide nanomaterial is 20% to 40% and a proportion of oxygen vacancies in the black bismuth oxyhalide nanomaterial is greater than 40%.

### Example 27

Preparation of a Grey Bismuth Oxyhalide Material with a Small Number of Oxygen Vacancies and a Black Bismuth Oxyhalide Nanomaterial with a Large Number of Oxygen Vacancies

(27-2-1) 0.97 g of Bi(NO₃)₃.5H₂O and 0.315 g of BiCl₃ were weighed and dissolved in 40 mL of DEG, and a resulting mixed solution was continuously stirred for 10 min on a magnetic stirrer at 80 r/min and then subjected to an ultrasonic treatment for 10 min in an ultrasonic machine to obtain a solution d.

(27-2-2) The solution d was transferred to a PTFE-lined hydrothermal reactor to undergo a reaction at 180° C. for 6 h.

(27-2-3) After the reaction was completed, a resulting reaction solution was cooled to room temperature, a resulting supernatant was removed, and a resulting precipitate was washed 4 times with water and ethanol and then dried to obtain a grey bismuth oxyhalide material e with a small number of oxygen vacancies for later use, which was denoted as sample 27-2.

(27-2-4) 50 mg of the grey bismuth oxyhalide material e with a small number of oxygen vacancies was taken and mixed with 2 g of SBH, and a resulting powdery mixture was transferred to a tube furnace and calcined at 400° C. for 2 h to obtain a bismuth oxyhalide nanomaterial d with a large number of oxygen vacancies.

(27-2-5) The bismuth oxyhalide nanomaterial d was washed 8 times with water and ethanol to obtain a solid, which was denoted as sample 27-3; the solid was dispersed in 10 mL of ethanol to obtain a dispersion.

(27-2-6) 10 mL of a solution of PEG in ethanol (with a concentration of 25 mg/mL) was added to the dispersion obtained in step (5), and a resulting mixed solution was continuously stirred for 24 h on a magnetic stirrer at 80 r/min. After a reaction was completed, a resulting mixture was centrifuged at 10,000 r/min, and a resulting precipitate was washed 4 times with water and ethanol to obtain a black 2D bismuth oxyhalide nanomaterial with a large number of oxygen vacancies, which was denoted as sample 27-4, and stored at 4° C.

Results

The TEM, DPBF degradation rate, photothermal conversion efficiency, cytotoxicity, CT value measurement, and in vivo CT imaging test results of the 2D bismuth oxyhalide composites with different numbers of oxygen vacancies obtained in Example 27 are basically the same as those of Example 1. XPS shows that a proportion of oxygen vacancies in the grey bismuth oxyhalide nanomaterial is 20% to 40% and a proportion of oxygen vacancies in the black bismuth oxyhalide nanomaterial is greater than 40%.

### Example 28

Preparation of a Grey Bismuth Oxyhalide Material with a Small Number of Oxygen Vacancies and a Black Bismuth Oxyhalide Nanomaterial with a Large Number of Oxygen Vacancies

(28-2-1) 0.97 g of Bi(NO₃)₃.5H₂O and 0.315 g of BiCl₃ were weighed and dissolved in 40 mL of DEG, and a resulting mixed solution was continuously stirred for 10 min on a magnetic stirrer at 80 r/min and then subjected to an ultrasonic treatment for 10 min in an ultrasonic machine to obtain a solution d.

(28-2-2) The solution d was transferred to a PTFE-lined hydrothermal reactor to undergo a reaction at 140° C. for 6 h.

(28-2-3) After the reaction was completed, a resulting reaction solution was cooled to room temperature, a resulting supernatant was removed, and a resulting precipitate was washed 4 times with water and ethanol and then dried to obtain a grey bismuth oxyhalide material e with a small number of oxygen vacancies for later use, which was denoted as sample 28-2.

(28-2-4) 50 mg of the grey bismuth oxyhalide material e with a small number of oxygen vacancies was taken and mixed with 4 g of SBH, and a resulting powdery mixture was transferred to a tube furnace and calcined at 400° C. for 2 h to obtain a bismuth oxyhalide nanomaterial d with a large number of oxygen vacancies.

(28-2-5) The bismuth oxyhalide nanomaterial d was washed 8 times with water and ethanol to obtain a solid, which was denoted as sample 28-3; the solid was dispersed in 10 mL of ethanol to obtain a dispersion.

(28-2-6) 10 mL of a solution of PLGA in ethanol (with a concentration of 25 mg/mL) was added to the dispersion obtained in step (5), and a resulting mixed solution was continuously stirred for 24 h on a magnetic stirrer at 80 r/min. After a reaction was completed, a resulting mixture was centrifuged at 10,000 r/min, and a resulting precipitate was washed 4 times with water and ethanol to obtain a black 2D bismuth oxyhalide nanomaterial with a large number of oxygen vacancies, which was denoted as sample 28-4 and stored at 4° C.

Results

The TEM, DPBF degradation rate, photothermal conversion efficiency, cytotoxicity, CT value measurement, and in vivo CT imaging test results of the 2D bismuth oxyhalide composites with different numbers of oxygen vacancies obtained in Example 28 are basically the same as those of Example 1. XPS shows that a proportion of oxygen vacancies in the grey bismuth oxyhalide nanomaterial is 20% to 40% and a proportion of oxygen vacancies in the black bismuth oxyhalide nanomaterial is greater than 40%.

### Example 29

Preparation of a Grey Bismuth Oxyhalide Material with a Small Number of Oxygen Vacancies and a Black Bismuth Oxyhalide Nanomaterial with a Large Number of Oxygen Vacancies

(29-2-1) 0.97 g of Bi(NO₃)₃.5H₂O and 0.315 g of BiCl₃ were weighed and dissolved in 40 mL of DEG, and a resulting mixed solution was continuously stirred for 10 min on a magnetic stirrer at 80 r/min and then subjected to an ultrasonic treatment for 10 min in an ultrasonic machine to obtain a solution d.

(29-2-2) The solution d was transferred to a PTFE-lined hydrothermal reactor to undergo a reaction at 140° C. for 6 h.

(29-2-3) After the reaction was completed, a resulting reaction solution was cooled to room temperature, a resulting supernatant was removed, and a resulting precipitate was washed 4 times with water and ethanol and then dried to obtain a grey bismuth oxyhalide material e with a small number of oxygen vacancies for later use, which was denoted as sample 29-2.

(29-2-4) 50 mg of the grey bismuth oxyhalide material e with a small number of oxygen vacancies was taken and mixed with 8 g of SBH, and a resulting powdery mixture was transferred to a tube furnace and calcined at 400° C. for 2 h to obtain a bismuth oxyhalide nanomaterial d with a large number of oxygen vacancies.

(29-2-5) The bismuth oxyhalide nanomaterial d was washed 8 times with water and ethanol to obtain a solid, which was denoted as sample 29-3; the solid was dispersed in 10 mL of ethanol to obtain a dispersion.

(29-2-6) 10 mL of a solution of PLGA in ethanol (with a concentration of 25 mg/mL) was added to the dispersion obtained in step (5), and a resulting mixed solution was continuously stirred for 24 h on a magnetic stirrer at 80 r/min. After a reaction was completed, a resulting mixture was centrifuged at 10,000 r/min, and a resulting precipitate was washed 4 times with water and ethanol to obtain a black 2D bismuth oxyhalide nanomaterial with a large number of oxygen vacancies, which was denoted as sample 29-4, and stored at 4° C.

Results

The TEM, DPBF degradation rate, photothermal conversion efficiency, cytotoxicity, CT value measurement, and in vivo CT imaging test results of the 2D bismuth oxyhalide composites with different numbers of oxygen vacancies obtained in Example 29 are basically the same as those of Example 1. XPS shows that a proportion of oxygen vacancies in the grey bismuth oxyhalide nanomaterial is 20% to 40% and a proportion of oxygen vacancies in the black bismuth oxyhalide nanomaterial is greater than 40%.

### Example 30

Preparation of a Grey Bismuth Oxyhalide Material with a Small Number of Oxygen Vacancies and a Black Bismuth Oxyhalide Nanomaterial with a Large Number of Oxygen Vacancies

(30-2-1) 0.97 g of Bi(NO₃)₃.5H₂O and 0.315 g of BiCl₃ were weighed and dissolved in 40 mL of DEG, and a resulting mixed solution was continuously stirred for 10 min on a magnetic stirrer at 80 r/min and then subjected to an ultrasonic treatment for 10 min in an ultrasonic machine to obtain a solution d.

(30-2-2) The solution d was transferred to a PTFE-lined hydrothermal reactor to undergo a reaction at 140° C. for 6 h.

(30-2-3) After the reaction was completed, a resulting reaction solution was cooled to room temperature, a resulting supernatant was removed, and a resulting precipitate was washed 4 times with water and ethanol and then dried to obtain a grey bismuth oxyhalide material e with a small number of oxygen vacancies for later use, which was denoted as sample 30-2.

(30-2-4) 50 mg of the grey bismuth oxyhalide material e with a small number of oxygen vacancies was taken and mixed with 8 g of SBH, and a resulting powdery mixture was transferred to a tube furnace and calcined at 400° C. for 4 h to obtain a bismuth oxyhalide nanomaterial d with a large number of oxygen vacancies.

(30-2-5) The bismuth oxyhalide nanomaterial d was washed 8 times with water and ethanol to obtain a solid, which was denoted as sample 30-3; the solid was dispersed in 10 mL of ethanol to obtain a dispersion.

(30-2-6) 10 mL of a solution of PVP in DEG (with a concentration of 25 mg/mL) was added to the dispersion obtained in step (5), and a resulting mixed solution was continuously stirred for 24 h on a magnetic stirrer at 80 r/min. After a reaction was completed, a resulting mixture was centrifuged at 10,000 r/min, and a resulting precipitate was washed 4 times with water and ethanol to obtain a black 2D bismuth oxyhalide nanomaterial with a large number of oxygen vacancies, which was denoted as sample 30-4, and stored at 4° C.

Results

The TEM, DPBF degradation rate, photothermal conversion efficiency, cytotoxicity, CT value measurement, and in vivo CT imaging test results of the 2D bismuth oxyhalide composites with different numbers of oxygen vacancies obtained in Example 30 are basically the same as those of Example 1. XPS shows that a proportion of oxygen vacancies in the grey bismuth oxyhalide nanomaterial is 20% to 40% and a proportion of oxygen vacancies in the black bismuth oxyhalide nanomaterial is greater than 40%.

What is claimed is:

1. A nanocomposite, comprising an oxygen vacancy-containing BiOX particle and a coating, wherein the coating is a biocompatible material;
under a near-infrared (NIR) irradiation, the nanocomposite has a photothermal conversion efficiency of greater than or equal to 10%;
under the NIR irradiation, the nanocomposite degrades 1,3-diphenylisobenzofuran (DPBF) at a rate of higher than or equal to 0.1 mmol/h; and
BiOX is at least one selected from the group consisting of BiOF, BiOCl, BiOBr, BiOI, and BiOAt.

2. The nanocomposite according to claim 1, wherein a proportion of an oxygen vacancy in the oxygen vacancy-containing BiOX particle is 20% or higher;
preferably, the proportion of the oxygen vacancy in the oxygen vacancy-containing BiOX particle is 20% to 30%;
preferably, the proportion of the oxygen vacancy in the oxygen vacancy-containing BiOX particle is 40% or higher;
preferably, under an NIR-II irradiation, the nanocomposite has the photothermal conversion efficiency of greater than or equal to 10%;
preferably, under the NIR-II irradiation, the nanocomposite has the photothermal conversion efficiency of greater than or equal to 40%;
preferably, under the NIR-II irradiation, the nanocomposite degrades the DPBF at the rate of higher than or equal to 1 mmol/h;
preferably, the nanocomposite has a computed tomography (CT) signal grey value of greater than or equal to 100;
preferably, the nanocomposite has a photoacoustic imaging (PAI) signal value of greater than or equal to 100.

3. (canceled)

4. (canceled)

5. (canceled)

6. (canceled)

7. (canceled)

8. (canceled)

9. (canceled)

10. The nanocomposite according to claim 1, wherein the oxygen vacancy-containing BiOX particle has a particle size of greater than or equal to 0.1 nm;
preferably, the oxygen vacancy-containing BiOX particle has the particle size of 0.1 nm to 500 nm.

11. (canceled)

12. The nanocomposite according to claim 1, wherein when BiOX is BiOCl, BiOCl particles with different numbers of oxygen vacancies are two-dimensional (2D) layered crystals.

13. The nanocomposite according to claim 1, wherein the coating is at least one selected from the group consisting of a siloxane polymer, a polysaccharide, a derivative of the polysaccharide, an amino acid, a derivative of the amino acid, a polyol, a derivative of the polyol, a polymer polyol, polyacrylic acid (PAA), and a derivative of the PAA.

14. The nanocomposite according to claim 13, wherein the coating is at least one selected from the group consisting of polyethylene glycol (PEG), a derivative of the PEG, mannitol, modified chitosan, dextran, carboxyl dextran, liposome, albumin, tetraethylorthosilicate (TEOS), the PAA, meglumine, arginine, polyglutamic acid (PGA), and polypeptide.

15. The nanocomposite according to claim 1, wherein a mass ratio of the oxygen vacancy-containing BiOX particle to the coating is 100:1 to 1:1.

16. A preparation method of the nanocomposite according to claim 1, comprising the following steps:
a) acquiring the oxygen vacancy-containing BiOX particle; and
b) coating the oxygen vacancy-containing BiOX particle to obtain the nanocomposite.

17. The preparation method of the nanocomposite according to claim 16, comprising the following steps:
a1) thoroughly mixing a Bi-containing oxycompound, a Bi-containing halide, and a solvent, and allowing a solvothermal reaction to produce a first oxygen vacancy-containing BiOX particle, wherein a proportion of an oxygen vacancy in the first oxygen vacancy-containing BiOX particle is 20% to 30%; and
b) mixing a first dispersion of the first oxygen vacancy-containing BiOX particle with a coating-containing solution or a coating precursor-containing solution, and allowing a reaction to produce the nanocomposite.

18. The preparation method of the nanocomposite according to claim 16, comprising the following steps:
a1) thoroughly mixing a Bi-containing oxycompound, a Bi-containing halide, and a solvent, and allowing a solvothermal reaction to produce a first oxygen vacancy-containing BiOX particle, wherein a proportion of an oxygen vacancy in the first oxygen vacancy-containing BiOX particle is 20% to 30%;
a2) subjecting a first dispersion of the first oxygen vacancy-containing BiOX particle to a reduction treatment to produce a second dispersion of a second oxygen vacancy-containing BiOX particle, wherein a proportion of an oxygen vacancy in the second oxygen vacancy-containing BiOX particle is 40% or higher; and
b) mixing the second dispersion with a coating-containing solution or a coating precursor-containing solution, and allowing a reaction to obtain the nanocomposite.

19. The preparation method of the nanocomposite according to claim 17, wherein in step a1), a mass ratio of the Bi-containing oxycompound to the Bi-containing halide is (10-1):(0.1-1).

20. The preparation method of the nanocomposite according to claim 17, wherein in step a1),
the Bi-containing oxycompound is at least one selected from the group consisting of Bi₂O₃, Bi₂(SO₄)₃, Bi(NO₃)₃.5H₂O, BiPO₄, BiH(PO₃)₂, BiH₂PO₃, Bi₂(CO₃)₃, Bi₂(SO₄)₃, and BiFeO₃;
the Bi-containing halide is at least one selected from the group consisting of BiF₃, BiCl₃, BiBr₃, BiI₃, and BiAt₃; and
the solvent is at least one selected from the group consisting of methanol, formaldehyde, ethanol, acetaldehyde, ethylene glycol (EG), diethylene glycol (DEG), dimethylformamide (DMF), benzyl alcohol, hydrazine hydrate, sodium borohydride (SBH), hydroiodic acid, acetone, dichloromethane (DCM), and trichloromethane (TCM).

21. The preparation method of the nanocomposite according to claim 17, wherein in step a1), the solvothermal reaction is conducted at 80° C. to 180° C. for 6 h to 48 h.

22. The preparation method of the nanocomposite according to claim 18, wherein in step a2), the reduction treatment comprises an ultraviolet (UV) light treatment or a reducing agent treatment.

23. The preparation method of the nanocomposite according to claim 22, wherein the UV light treatment is conducted at 10 W to 500 W for 2 h to 12 h.

24. The preparation method of the nanocomposite according to claim 22, wherein the reducing agent treatment comprises calcining the first dispersion of the first oxygen vacancy-containing BiOX particle in the presence of a reducing agent at 300° C. to 400° C. for 2 h to 12 h.

25. The preparation method of the nanocomposite according to claim 24, wherein the reducing agent is at least one selected from the group consisting of SBH, potassium borohydride (KBH), stannous chloride, oxalic acid, and dithizone.

26. The preparation method of the nanocomposite according to claim 24, wherein a mass ratio of the reducing agent to the first oxygen vacancy-containing BiOX particle is 1:(100-1).

27. The preparation method of the nanocomposite according to claim 17, wherein in step b), the reaction is conducted at 20° C. to 35° C. under stirring.

28. The nanocomposite according to claim 1, wherein the nanocomposite is used in at least one of the following: a preparation of a nanomaterial for photothermal therapy (PTT) of a tumor, a preparation of a nanomaterial for photodynamic therapy (PDT) of the tumor, a preparation of a tumor-targeted drug, a preparation of a material for tumor diagnosis, a preparation of a material for tumor diagnosis in vitro and in vivo, a cell isolation, a drug carrier, a preparation of a material for heavy-ion therapy, a preparation of a material for isotope diagnosis and treatment, and a preparation of a material for integrated tumor diagnosis and treatment.

29. (canceled)

30. (canceled)

31. (canceled)

32. (canceled)

33. (canceled)

34. (canceled)

35. (canceled)

36. (canceled)

37. (canceled)

38. (canceled)
